(19) United States
(12) Patent Application Publication
(10) Pub. No.: US 2024/0076626 A1
(43) Pub. Date: Mar. 7, 2024

(54) HYDROGEN PEROXIDE EVOLVED HOST CELL

(71) Applicant: MedImmune Limited, Cambridge (GB)

(72) Inventors: Rajesh Kumar MISTRY, Cambridge (GB); Emma Jane KELSALL, Cambridge (GB); Si Nga SOU, Cambridge (GB); Suzanne GIBSON, Cambridge (GB); Diane HATTON, Cambridge (GB); Michal JENNS, Cambridge (GB); Katie WILLIS, Cambridge (GB)

(21) Appl. No.: 18/256,768

(22) PCT Filed: Dec. 10, 2021
(86) PCT No.: PCT/IB2021/061570

§ 371 Date: Jun. 9, 2023

Related U.S. Application Data
(60) Provisional application No. 63/199,177, filed on Dec. 11, 2020.

(51) Int. Cl. C12N 5/071

(52) U.S. Cl. CPC C12N 5/0682; C12N 2510/00

(57) ABSTRACT

A directed evolution method for improving performance of a mammalian host cell is provided, as well as host cells generated using the directed evolution method. In one aspect, hydrogen peroxide (H₂O₂)-evolved host cells are provided. In one aspect, (H₂O₂)-evolved Chinese hamster ovary (CHO) host cells are provided.

### RELATED APPLICATIONS

This application claims the benefit of priority under 35 USC 119(e) to U.S. Provisional Application No. 63/199,177, filed on Dec. 11, 2020. The entire contents of the foregoing are hereby incorporated by reference.

### FIELD OF THE INVENTION

The present disclosure relates to a hydrogen peroxide (H₂O₂)-evolved host cell and methods for making and using the (H₂O₂)-evolved host cell, and in particular, to methods of producing of a protein of interest using the (H₂O₂)-evolved host cell.

### BACKGROUND OF THE INVENTION

More than 100 therapeutic proteins are approved for clinical use in the European Union and the USA, most of which are recombinantly produced. (Dimitrov, DS (2012) “Therapeutic Proteins.” Methods Mol. Biol. 899:1-26). Engineered proteins with non-native formats, such as bispecific antibodies, multispecific fusion proteins, and antibody conjugates are also being developed. Despite the huge success of protein-based therapeutics, efficient development remains challenging. In particular, protein overexpression can impact host cell metabolism and large-scale cell culture can result in cellular stress, reducing host cell productivity performance.

Reactive oxygen species (ROS) are natural byproducts of aerobic metabolism which can interfere with cell growth and productivity when present at high levels. (Chevallier et al. (2019) “Oxidative stress-alleviating strategies to improve recombinant protein production in CHO cells”. Biotechnol Bioeng. 117(4):1172-1186; Zeeshan et al. (2016) “Endoplasmic Reticulum Stress and Associated ROS”. Int J Mol Sci. 17(3):327; Turrens J F. (2003) “Mitochondrial formation of reactive oxygen species”. J Physiol. 552(Pt 2):335-44; Santos et al. (2009) “Mechanisms and implications of reactive oxygen species generation during the unfolded protein response: roles of endoplasmic reticulum oxidoreductases, mitochondrial electron transport, and NADPH oxidase”. Antioxid Redox Signal. 11(10):2409-27). Awareness of the impact of cellular redox state on recombinant protein production has increased in recent years. (Handlogten et al. (2017) “Glutathione and thioredoxin systems contribute to recombinant monoclonal antibody interchain disulfide bond reduction during bioprocessing”. Biotechnol Bioeng. 114(7):1469-77; Handlogten, et al. (2018) “Intracellular response to process optimization and impact on productivity and product aggregates for a high-titer CHO cell process”. Biotechnol Bioeng. 115(1):126-38; Orellana et al. (2015) “High-antibody-producing Chinese hamster ovary cells up-regulate intracellular protein transport and glutathione synthesis”. J Proteome Res. 14(2):609-18; and Handlogten et al. (2020) “Online Control of Cell Culture Redox Potential Prevents Antibody Interchain Disulfide Bond Reduction”. Biotechnol Bioeng. 117(5):1329-1336).

Chinese Hamster Ovary (CHO) cells are frequently used for expression of monoclonal antibodies (mAb) and other non-native biopharmaceutical molecules such as bispecific antibodies (BisAbs). (Kim et al. (2012) “CHO cells in biotechnology for production of recombinant proteins: current state and further potential”. Appl Microbiol Biotechnol. 93(3):917-30; and Wang, et al. (2019) “Design and Production of Bispecific Antibodies”. Antibodies. 8(3):43. However, CHO cells are believed to produce high levels of ROS during the large-scale cell culture which can lead to oxidative stress, suboptimal cell culture performance and lower antibody titers. (Handlogten et al. (2018) “Intracellular response of CHO cells to oxidative stress and its influence on metabolism and antibody production”. Biochemical Engineering Journal. 133:12-20). In particular, development of novel antibody formats, such as, bispecific antibodies, can be hindered by low product yields (Spiess et al. (2015) “Alternative molecular formats and therapeutic applications for bispecific antibodies”. Mol Immunol. 67(2 Pt A):95-106) associated with high levels of cellular stress, including oxidative stress (Chevallier et al. (2019) “Oxidative stress-alleviating strategies to improve recombinant protein production in CHO cells”. Biotechnol Bioeng. 117(4):1172-11).

Genetic engineering approaches involving manipulation of specific genes through overexpression or targeted genetic ablation to alter subcellular processes have been implemented to relieve production bottlenecks and produce more predictable and robust cell lines. To date, this strategy has been employed to alter diverse subcellular processes including cell cycle (Fussenegger et al. (1998) “Controlled proliferation by multigene metabolic engineering enhances the productivity of Chinese hamster ovary cells”. Nat Biotechnol. 16(5):468-72), metabolism (Fogolin et al. (2004) “Impact of temperature reduction and expression of yeast pyruvate carboxylase on hGM-CSF-producing CHO cells”. J Biotechnol. 109(1-2):179-91), protein secretion (Mohan et al. (2007) “Effect of doxycycline-regulated protein disulfide isomerase expression on the specific productivity of recombinant CHO cells: thrombopoietin and antibody”. Biotechnol Bioeng. 98(3):611-5) and cellular redox (Orellana et al. (2017) “Overexpression of the regulatory subunit of glutamate-cysteine ligase enhances monoclonal antibody production in CHO cells”. Biotechnol Bioeng. 114(8):1825-36; Banmeyer et al. (2004) “Overexpression of human peroxiredoxin 5 in subcellular compartments of Chinese hamster ovary cells: effects on cytotoxicity and DNA damage caused by peroxides”. Free Radic Biol Med. 36(1):65-77; Warner et al. (1993) “Expression of human Mn SOD in Chinese hamster ovary cells confers protection from oxidant injury”. Am J Physiol. 264(6 Pt 1):L598-605).

Although these genetic engineering strategies have met with moderate success, targeting specific biochemical pathways to manipulate specific genes may not be as effective as originally hypothesized. Thus, there remains a need for cell lines with improved performance during cell culture, for example, when expressing a heterologous protein, or a protein with a non-native format such as a bispecific antibody.

### SUMMARY OF THE INVENTION

The present invention relates to methods for producing a recombinant protein of interest, including, for example, an antibody, an antigen-binding antibody fragment, or a bispecific antibody, and, in particular, to methods of producing a recombinant protein of interest using a hydrogen-peroxide (H₂O₂)-evolved mammalian host cell.

In one aspect, a hydrogen peroxide (H₂O₂)-evolved mammalian host cell is provided that is capable expressing a protein of interest. In one aspect, the H₂O₂-evolved host cell has an antioxidant defense system in which a level of one or more components of the antioxidant defense system are increased when compared to a parental control. In one aspect, one or more components of the antioxidant defense system are selected from glutathione (GSH), oxidized glutathione (GSSG), glutathione synthetase (GSS); glutamate cysteine ligase modifier subunit (GCLM); catalase; cysteine/glutamate antiporter light chain (xCT); glutathione peroxidase-1 (GPx-1); and combinations thereof. In one aspect, the H₂O₂-evolved host cell has an increased resistance to cellular stress when compared to a parental control. In one aspect, the H₂O₂-evolved host cell has an increased resistance to oxidative stress when compared to a parental control.

In one aspect, the H₂O₂-evolved host cell has an increased level of total glutathione when compared to the parental control. In one aspect, the H₂O₂-evolved host cell has from about a 1% to about 25%, about 2% to about 20%, or about 3% to about 10%, or at least about 1%, about 2%, about 3%, about 4%, about 5%, about 6%, about 7%, about 8%, about 9%, about 10%, about 15%, or about 20% higher level of GSH than the parental control. In one aspect, the H₂O₂-evolved host cell has an increased ratio of total glutathione to oxidized glutathione (GSSG) (GSH:GSSG) when compared to a parental control. In one aspect, the ratio of total glutathione to GSSG (GSH:GSSG) is increased by about 1% to about 15%, or about 2% to about 10%, or at least about 1%, about 2%, about 3%, about 4%, about 5%, about 6%, about 7%, about 8%, about 9%, about 10%, or about 15% as compared to the parental control. In one aspect, the ratio of total glutathione to GSSG (GSH:GSSG) is from about 2.5:1 to about 3:1, or at least about 2.5:1, about 2.6:1, about 2.7:1, about 2.8:1, about 2.9:1 or about 3:1.

In one aspect, one or more antioxidant defense genes of the H₂O₂-evolved host cell are upregulated as compared to a parental control. In one aspect, one or more antioxidant defense genes are selected from: glutathione synthetase (GSS); glutamate cysteine ligase modifier subunit (GCLM); catalase; cysteine/glutamate antiporter light chain (xCT); glutathione peroxidase-1 (GPx-1); and combinations thereof. In one aspect, GSS expression is increased about 10% to about 300%, or about 25% to about 200%, or at least about 10%, about 20%, about 30%, about 40%, about 50%, about 60%, about 70%, about 80%, about 90%, about 100% and up to about 150%, about 200%, about 250% or about 300% as compared to the parental control. In one aspect, GCLM expression is increased about 10% to about 100%, or about 25% to about 75%, or at least about 10%, about 15%, about 20%, about 25%, about 30%, about 35%, about 40%, about 45%, about 50%, about 55%, about 60%, about 65%, about 70%, or about 75% as compared to the parental control. In one aspect, catalase expression is increased about 10% to about 100%, or about 25% to about 75%, or at least about 10%, about 15%, about 20%, about 25%, about 30%, about 35%, about 40%, about 45%, about 50%, about 55%, about 60%, about 65%, about 70%, or about 75% as compared to the parental control. In one aspect, xCT expression is increased about 10% to about 50%, or at least about 10%, about 15%, about 20%, about 25%, about 30%, about 35%, about 40%, about 45%, or about 50% as compared to the parental control. In one aspect, GPx-1 expression is increased about 10% to about 100%, or about 10% to about 50%, or at least about 10%, about 15%, about 20%, about 25%, about 30%, about 35%, about 40%, about 45%, or about 50% as compared to the parental control.

In one aspect, the H₂O₂-evolved host cell is a Chinese hamster ovary (CHO) cell.

In one aspect, the H₂O₂-evolved host cell includes a heterologous gene encoding a protein of interest. In one aspect, the heterologous gene is stably integrated into host cell DNA. In one aspect, the heterologous gene encodes an antibody or an antigen-binding antibody fragment. In one aspect, the heterologous gene encodes a bispecific antibody.

In one aspect, a hydrogen peroxide (H₂O₂)-evolved Chinese hamster ovary (CHO) cell is provided that is capable of expressing a therapeutic protein of interest. In one aspect, the H₂O₂-evolved host cell has an antioxidant defense system in which a level of one or more components of the antioxidant defense system selected from glutathione (GSH), oxidized glutathione (GSSG), glutathione synthetase (GSS); glutamate cysteine ligase modifier subunit (GCLM); catalase; cysteine/glutamate antiporter light chain (xCT); glutathione peroxidase-1 (GPx-1); or a combination thereof, are increased when compared to a parental control.

In one aspect, a population of cells is provided that include a H₂O₂-evolved host cell described herein. In one aspect, the population of H₂O₂-evolved cells have improved performance as compared to a population of parental control cells. In one aspect, the H₂O₂-evolved cells have an improved performance that includes one or more of:
(a) increased viable cell density;
(b) increased viability;
(c) decreased lactate levels;
(d) increased titer;
(e) increased specific productivity (qP);
or a combination thereof.

In one aspect, the population of cells include H₂O₂-evolved cells with improved performance as compared to a population of parental control cells when cultured in a fed-batch culture process. In one aspect, the population of cells include H₂O₂-evolved cells with improved performance as compared to a population of parental control cells when expressing a protein of interest. In one aspect, the population of cells express a protein of interest that is a bispecific antibody.

In one aspect, the population of H₂O₂-evolved cells has an increased viable cell density as compared to the population of parental control cells. In one aspect, the population of H₂O₂-evolved cells has a peak viable cell density from about 15×10⁶ cells/mL to about 25×10⁶ cells/mL, or at least about 15×10⁶ cells/mL, about 16×10⁶ cells/mL, about 17×10⁶ cells/mL, about 18×10⁶ cells/mL, or about 19×10⁶ cells/mL, and up to about 20×10⁶ cells/mL.

In one aspect, the population of H₂O₂-evolved cells have reduced lactate levels as compared to the population of parental control cells. In one aspect, the lactate accumulation for the population of H₂O₂-evolved cells is less than about 75%, about 70%, about 65%, about 60%, about 55%, about 50%, about 45%, about 40%, about 35%, about 30%, about 25% or about 20% of the lactate accumulation of a non-evolved parental control culture.

In one aspect, the population of cells express a protein of interest at a titer that is increased at least about 1.5 fold to 3.5 fold, or at least about 1.5 fold, about 1.75 fold, about 2.0 fold, about 2.25 fold, about 2.5 fold, about 2.75 fold, about 3.0 fold, about 3.25 fold, or about 3.5 fold as compared to a population of parental control cells. In one aspect, the population of cells express a protein of interest that is a bispecific antibody. In one aspect, the titer of the bispecific antibody is from about 0.5 g/L and about 1.5 g/L, or about 0.5 g/L and about 1.1 g/L, or from about 0.50 g/L, about 0.55 g/L, about 0.60 g/L, about 0.65 g/L, about 0.70 g/L, about 0.75 g/L, about 0.80 g/L, about 0.85 g/L, about 0.90 g/L, about 0.95 g/L, and up to about 1.0 g/L, about 1.1 g/L, about 1.2 g/L, about 1.3 g/L, about 1.4 g/L, or about 1.5 g/L. In one aspect, the titer of the bispecific antibody is at least about 0.5 g/L, about 0.6 g/L, about 0.7 g/L, about 0.8 g/L, about 0.9 g/L, or about 1.0 g/L and up to about 1.0 g/L.

In one aspect, the population of H₂O₂-evolved cells have a specific productivity (qP) that is increased at least about 1.1 fold, about 1.2 fold, about 1.3 fold, about 1.4 fold, about 1.5 fold, about 1.6 fold, about 1.7 fold, about 1.8 fold, about 1.9 fold, or about 2.0 fold as compared to the population of parental control cells.

In one aspect, a method of producing a protein of interest is provided. In one aspect, the method includes culturing a H₂O₂-evolved host cell as described herein under conditions that allow for expression of the protein of interest. In one aspect, the protein of interest is a heterologous protein. In one aspect, the protein of interest is an antibody or an antigen-binding antibody fragment. In one aspect, the antibody is a bispecific antibody. In one aspect, the method includes isolating the protein of interest. In one aspect, a composition is provided that includes the isolated protein of interest and a pharmaceutically acceptable carrier.

In one aspect, provided herein is a use of a H₂O₂-evolved host cell as described herein for producing a protein of interest. In one aspect, the protein of interest is an antibody or an antigen-binding antibody fragment. In one aspect, the antibody is a bispecific antibody.

In one aspect, an isolated host cell is provided that has improved viability in the presence of hydrogen peroxide (H₂O₂). In one aspect, the host cell has a viability of at least about 10%, about 15%, about 20%, about 25%, about 30%, about 35%, about 40%, about 45%, or about 50% viability after about 1 to about 15 days after exposure to about 2 mM to about 50 mM H₂O₂, about 10 mM to about 40 mM H₂O₂, about 20 mM to about 40 mM H₂O₂, about 30 mM to about 40 mM H₂O₂, or about 35 mM to about 40 mM H₂O₂. In one aspect, the host cell is contacted with at least about 10 mM, about 15 mM, about 20 mM, about 25 mM, about 30 mM, about 35 mM and up to about 40 mM, about 45 mM or about 50 mM H₂O₂. In one aspect, the host cell is contacted with about 30 mM, about 31 mM, about 32 mM, about 33 mM, about 34 mM, about 35 mM, about 36 mM, about 37 mM, about 38 mM, about 39 mM or about 40 mM H₂O₂.

In one aspect, the population of H₂O₂-evolved cells has at least about 10%, about 20%, about 30%, about 40%, or about 50% increased viability as compared to a population of parental control cells when challenged with hydrogen peroxide. In one aspect, the population of H₂O₂-evolved cells are challenged with at least about 5 mM, about 10 mM, about 15 mM, about 20 mM, about 25 mM, about 30 mM or about 35 mM, and up to about 40 mM hydrogen peroxide. In one aspect, the population of H₂O₂-evolved cells are challenged with about 30 mM, about 31 mM, about 32 mM, about 33 mM, about 34 mM, about 35 mM, about 36 mM, about 37 mM, about 38 mM, about 39 mM, or about 40 mM hydrogen peroxide. In one aspect, the improved viability of the H₂O₂-evolved cells after challenge with hydrogen peroxide is maintained over at least about 50, about 60, about 70, about 80, about 90, or about 100 population doublings (PDL).

In one aspect, a method is provided for producing an evolved population of mammalian host cells with increased resistance to cellular stress. In one aspect, a method is provided for producing an evolved population of mammalian host cells with increased resistance to oxidative stress. In one aspect, the method includes multiple rounds of culturing the population of cells in the presence of about 5 mM to about 20 mM, or about 10 mM to about 15 mM hydrogen peroxide (H₂O₂) and allowing the cells to recover until cells can survive in the presence of from about 20 mM to about 40 mM H₂O₂. In one aspect, the method includes:
(a) providing a population of cells;
(b) culturing the population of cells in a cell culture media;
(c) contacting the population of cells with about 5 mM to about 20 mM H₂O₂ to provide a population of transitional cells;
(d) resuspending the transitional cells in fresh cell culture media that does not include H₂O₂ and culturing until cells reach at least about 70% viability;
(e) repeating steps (c)-(d) to obtain a population of H₂O₂-evolved cells that can survive when contacted with about 20 mM to about 40 mM H₂O₂ and incubated for about 30 minutes to about 1 hour.

In one aspect, the population of cells in produced in (b) is cultured to at least about 70%, about 80% or about 90% viability. In one aspect, the population of cells in produced in (c) is contacted with about 5 mM to about 10 mM, about 10 to about 15 mM, or about 15 to about 20 mM H₂O₂. In one aspect, the population of cells in produced in (c) is contacted with about 10 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, and up to about 20 mM H₂O₂. In one aspect, the population of cells in produced in (c) comprises incubating the population of cells with H₂O₂ for at least about 30 min, about 45 min or about 60 min and up to about 90 min, or about 120 min. In one aspect, steps (c)-(d) are repeated at least about 3, about 4, or about 5 and up to about 6, about 7, about 8, about 9 or about 10 times.

In one aspect, a method is provided for producing an evolved population of Chinese hamster ovary (CHO) cells.

In one aspect, a method is provided for producing an evolved population of mammalian host cells that include a heterologous gene encoding a protein of interest. In one aspect, the heterologous gene is stably integrated into host cell DNA. In one aspect, the heterologous gene encodes an antibody or an antigen-binding antibody fragment. In one aspect, the heterologous gene encodes a bispecific antibody.

In one aspect, a method is provided for producing an evolved population of mammalian host cells, in which the cells have improved performance as compared to a non-evolved control when cultured in suspension.

In one aspect, the population of evolved cells can survive when contacted with about 20 mM to about 40 mM H₂O₂ after at least about 20, about 30, about 40, about 50, about 60, about 70, about 80, about 90, or about 100 and up to about 120 population doublings (PDL).

In one aspect, the cell culture media includes from about 1 mM to about 10 mM, or about 2 mM to about 8 mM, or about 4 mM to about 6 mM L-glutamine.

In one aspect, an evolved host cell is provided that is produced by the methods described herein.

In one aspect, an H₂O₂-evolved host cell is provided that is stably transfected with a heterologous gene encoding a protein of interest, wherein the host cell has a viability from about 70% to about 85% within about 9 to about 12 days post-transfection. In one aspect, the host cell has a viability from about 70% to about 85% at least about 9 days, about 10 days, about 11 days or about 12 days post-transfection. In one aspect, the host cell has viable cell density (VCD) from about 1.0×10⁶ cells/ml to about 1.6×10⁶ cells/ml at least about 9 days, 10 days, 11 days or 12 days post-transfection. In one aspect, the H₂O₂-evolved host cell is a Chinese hamster ovary (CHO) cell. In one aspect, stable transfection includes electroporation followed by methionine sulphoximine (MSX) selection. In one aspect, the heterologous gene encodes an antibody. In one aspect, the heterologous gene encodes a bispecific antibody.

### BRIEF DESCRIPTION OF THE FIGURES

FIG. 1A shows a viability plot tracking cell recovery during the H₂O₂ host evolution process (arrows indicate the day and concentration of H₂O₂ addition, black circles represent cell viability counts).

FIG. 1B shows the viability plot for CHO control host and H₂O₂ evolved host after challenge with 37 mM H₂O₂. The H₂O₂ evolved host and CHO control host were passaged to 90 PDL and 7 PDL respectively in CD-CHO supplemented with 6 mM glutamine after which both hosts were re-challenged with 37 mM H₂O₂ and viabilities recorded (viability measured on days 1, 2, 4, 7, 10, 11 and 12)

FIG. 2A is a graph showing a comparison of relative host cell viabilities following 72 hours incubation with Menadione Sodium Bisulphite (MSB) between CHO control and H₂O₂ evolved host cells. N=3, statistics determined using an unpaired t-test, *=P<0.05.

FIG. 2B is a graph showing a comparison of relative host cell viabilities following 72 hours incubation with Buthionine Sulfoximine (BSO) between CHO control and H₂O₂ evolved host cells. N=3, statistics determined using an unpaired t-test, *=P<0.05.

FIG. 2C is a graph showing a comparison of relative host cell viabilities following 72 hours incubation with Mercaptosuccinic Acid (MS) between CHO control and H₂O₂ evolved host cells. N=3, statistics determined using an unpaired t-test, *=P<0.05.

FIG. 2D is a graph showing a comparison of relative host cell viabilities following 72 hours incubation with Cobalt chloride (CoCl) between CHO control and H₂O₂ evolved host cells. N=3, statistics determined using an unpaired t-test, **=P<0.005.

FIG. 3A is a graph showing a comparison of total glutathione between untransfected CHO control and H₂O₂ evolved hosts. N=3, statistics determined using an unpaired t-test, ***=P<0.0005.

FIG. 3B is a graph showing a comparison of the ratio of total glutathione:GSSG (GSH:GSSG) between untransfected CHO control and H₂O₂ evolved hosts. N=3, statistics determined using an unpaired t-test.

FIG. 3C is a graph showing the relative mRNA expression of glutathione synthetase (GSS) in untransfected CHO control and H₂O₂ evolved host cells. All qPCR data was normalised to MMADHC mRNA expression. N=3, statistics determined using an unpaired t-test, **=P<0.005.

FIG. 3D is a graph showing the relative mRNA expression of Gamma Glutamyl Cysteine Ligase Modulator subunit (GCLM) in untransfected CHO control and H₂O₂ evolved host cells. All qPCR data was normalized to MMADHC mRNA expression. N=3, statistics determined using an unpaired t-test, ***=P<0.0005.

FIG. 3E is a graph showing the relative mRNA expression of Catalase in untransfected CHO control and H₂O₂ evolved host cells. All qPCR data was normalized to MMADHC mRNA expression. N=3, statistics determined using an unpaired t-test, ***=P<0.0005.

FIG. 3F is a graph showing the relative mRNA expression of xCT in untransfected CHO control and H₂O₂ evolved host cells. All qPCR data was normalized to MMADHC mRNA expression. N=3, statistics determined using an unpaired t-test, ***=P<0.0005.

FIG. 4A is a viability plot of CHO control host A and H₂O₂ evolved host A in response to 6 uM MSB or H₂O treatment for 72 hours. N=3, statistics determined using a one-way ANOVA and a Tukey's multiple comparison test, *=P<0.05 (compares CHO control host A+6 μM MSB and H₂O₂ evolved host A+6 μM MSB).

FIG. 4B is a viability plot of control host B and H₂O₂ evolved host B in response to 6 μM MSB or H₂O treatment for 72 hours. N=3, statistics determined using a one-way ANOVA and a Tukey's multiple comparison test, *=P<0.05 (compares CHO control host A+6 μM MSB and H₂O₂ evolved host A+6 μM MSB).

FIG. 5A shows a comparison of total glutathione in CHO control host (A) and H₂O₂ evolved host (A) expressing bispecific antibody A (BisAb AN=3, statistics determined using an unpaired t-test, ***=P<0.0005.

FIG. 5B shows a comparison of the ratio of total glutathione:GSSG (GSH:GSSG) in CHO control host (A) and H₂O₂ evolved host (A) expressing bispecific antibody A (BisAb A). N=3, statistics determined using an unpaired t-test, **=P<0.005.

FIG. 5C shows a comparison of relative mRNA expression of glutathione synthetase (GSS) in CHO control host (A) and H₂O₂ evolved host (A) expressing bispecific antibody A (BisAb A). All qPCR data was normalized to MMADHC mRNA expression. N=3, statistics determined using an unpaired t-test, ****=P<0.00005.

FIG. 5D shows a comparison of Gamma Glutamyl Cysteine Ligase Modulator subunit (GCLM) in CHO control host (A) and H₂O₂ evolved host (A) expressing bispecific antibody A (BisAb A). All qPCR data was normalized to MMADHC mRNA expression. N=3, statistics determined using an unpaired t-test, ****=P<0.00005.

FIG. 5E shows a comparison of catalase in CHO control host (A) and H₂O₂ evolved host (A) expressing bispecific antibody A (BisAb A). All qPCR data was normalized to MMADHC mRNA expression. N=3, statistics determined using an unpaired t-test, **=P<0.005.

FIG. 5F shows a comparison of xCT in CHO control host (A) and H₂O₂ evolved host (A) expressing bispecific antibody A (BisAb A). All qPCR data was normalized to MMADHC mRNA expression. N=3, statistics determined using an unpaired t-test, ***=P<0.0005.

FIG. 5G shows a comparison of Glutathione Peroxidasel (GPx1) in CHO control host (A) and H₂O₂ evolved host (A) expressing bispecific antibody A (BisAb A). All qPCR data was normalized to MMADHC mRNA expression. N=3, statistics determined using an unpaired t-test, **=P<0.005.

FIG. 6A shows a comparison of total glutathione in CHO control host (B) and H₂O₂ evolved host (B) expressing bispecific antibody B (BisAb B). N=3, statistics determined using an unpaired t-test, * *=P<0.005.

FIG. 6B shows a comparison of the ratio of total glutathione:GSSG (GSH:GSSG) in CHO control host (B) and H₂O₂ evolved host (B) expressing bispecific antibody B (BisAb B). N=3, statistics determined using an unpaired t-test, **=P<0.005.

FIG. 6C shows a comparison of relative mRNA expression of GSS in CHO control host (B) and H₂O₂ evolved host (B) expressing bispecific antibody B (BisAb B). All qPCR data was normalized to MMADHC mRNA expression. N=3, statistics determined using an unpaired t-test, ***=P<0.0005.

FIG. 6D shows a comparison of GCLM in CHO control host (B) and H₂O₂ evolved host (B) expressing bispecific antibody B (BisAb B). All qPCR data was normalized to MMADHC mRNA expression. N=3, statistics determined using an unpaired t-test, **=P<0.005.

FIG. 6E shows a comparison of catalase in CHO control host (B) and H₂O₂ evolved host (B) expressing bispecific antibody B (BisAb B). All qPCR data was normalized to MMADHC mRNA expression. N=3, statistics determined using an unpaired t-test, ***=P<0.0005.

FIG. 6F shows a comparison of xCT in CHO control host (B) and H₂O₂ evolved host (B) expressing bispecific antibody B (BisAb B). All qPCR data was normalized to MMADHC mRNA expression. N=3, statistics determined using an unpaired t-test.

FIG. 6G shows a comparison of GPx1 in CHO control host (B) and H₂O₂ evolved host (B) expressing bispecific antibody B (BisAb B). All qPCR data was normalized to MMADHC mRNA expression. N=3, statistics determined using an unpaired t-test.

FIG. 7A shows cell viability of CHO control host A and B and H₂O₂ evolved host A and B where monitored after transfection. Three pools were generated for each molecule and each host.

FIG. 7B shows viable cell density (VCD) of CHO control host A and B and H₂O₂ evolved host A and B where monitored after transfection. Three pools were generated for each molecule and each host.

FIG. 8A shows a comparison of viable cell density (VCD) (10⁶ cells/mL) between CHO control A and H₂O₂ evolved host A expressing bispecific antibody A (BisAb A). A total of 3 pools expressing each molecule were evaluated for each host.

FIG. 8B shows a comparison of viability (%) between CHO control A and H₂O₂ evolved host A bispecific antibody A (BisAb A). A total of 3 pools expressing each molecule were evaluated for each host.

FIG. 8C shows a comparison of lactate (g/L) between CHO control A and H₂O₂ evolved host A expressing bispecific antibody A (BisAb A). A total of 3 pools expressing each molecule were evaluated for each host.

FIG. 8D shows a comparison of titer between CHO control A and H₂O₂ evolved host A expressing bispecific antibody A (BisAb A). A total of 3 pools expressing each molecule were evaluated for each host. Statistics determined using an unpaired t-test, ****=P<0.00005.

FIG. 8E shows a comparison of cell specific productivity (qP) between CHO control A and H₂O₂ evolved host A expressing bispecific antibody A (BisAb A). A total of 3 pools expressing each molecule were evaluated for each host. Statistics determined using an unpaired t-test, *=P<0.05.

FIG. 9A shows a comparison of viable cell density (VCD) (10⁶ cells/mL) between CHO control B and H₂O₂ evolved host B expressing bispecific antibody B (BisAb B). A total of 3 pools expressing each molecule were evaluated for each host.

FIG. 9B shows a comparison of viability (%) between CHO control B and H₂O₂ evolved host B expressing bispecific antibody B (BisAb B). A total of 3 pools expressing each molecule were evaluated for each host.

FIG. 9C shows a comparison of lactate (g/L) between CHO control B and H₂O₂ evolved host B expressing bispecific antibody B (BisAb B). A total of 3 pools expressing each molecule were evaluated for each host.

FIG. 9D shows a comparison of titer between CHO control B and H₂O₂ evolved host B expressing bispecific antibody B (BisAb B). A total of 3 pools expressing each molecule were evaluated for each host. Statistics determined using an unpaired t-test, *=P<0.05.

FIG. 9E shows a comparison of cell specific productivity (qP) between CHO control B and H₂O₂ evolved host B expressing bispecific antibody B (BisAb B). A total of 3 pools expressing each molecule were evaluated for each host. Statistics determined using an unpaired t-test.

FIG. 10 is a flow chart of a method of directed evolution to generate hydrogen-peroxide evolved host cells.

FIG. 11 is a graph showing the transfection recovery of an H₂O₂-evolved host and a CHO host after transfection with linearized DNA encoding an IgG monoclonal antibody.

FIG. 12A shows viable cell density of a H₂O₂-evolved host expressing a murine IgG mAb and a CHO control.

FIG. 12B shows the % viability of a H₂O₂-evolved host expressing a murine IgG mAb and a CHO control.

FIG. 12C shows the titre (mg/L) of a H₂O₂-evolved host expressing a murine IgG mAb and a CHO control.

FIG. 12D shows the lactate profile (g/L) of a H₂O₂-evolved host expressing a murine IgG mAb and a CHO control.

FIG. 12E shows the specific productivity (qP) of a H₂O₂-evolved host expressing a murine IgG mAb and a CHO control.

### DETAILED DESCRIPTION OF THE INVENTION

### A. Definitions

Unless otherwise defined, scientific and technical terms used herein shall have the meanings that are commonly understood by those of ordinary skill in the art. Further, unless otherwise required by context, singular terms shall include pluralities and plural terms shall include the singular, for example, “a” or “an”, include pluralities, e.g., “one or more” or “at least one” and the term “or” can mean “and/or”, unless stated otherwise. The terms “including,” “includes” and “included”, are not limiting. Ranges provided herein, of any type, include all values within a particular range described and values about an endpoint for a particular range.

As used herein, the term “about” is used to modify, for example, the quantity of an ingredient in a composition, concentration, volume, process temperature, process time, yield, flow rate, pressure, and ranges thereof, employed in describing the invention. The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and other similar considerations. The term “about” also encompasses amounts that differ due to aging of a formulation with a particular initial concentration or mixture, and amounts that differ due to mixing or processing a formulation with a particular initial concentration or mixture. Where modified by the term “about,” the claims appended hereto include such equivalents.

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Cellular stress” refers to an environmental stressor that can impact performance or productivity of a host cell. Cellular stress can include transfection processes, for example transient or stable transfection. Cellular stress can be induced by recombinant expression of a heterologous protein, including, for example, a difficult to express protein such as a bispecific antibody. Cellular stress can also include cell culture processes such as a batch or fed-batch cell culture process.

“Oxidative stress” refers to an imbalance between generation of reactive oxygen species (ROS) produced by cells and the antioxidative capacity of the cells. Although ROS are natural byproducts of aerobic metabolism, they can adversely impact cell health and productivity at high levels. The term “resistant” or “resistance” to oxidative stress refers to the improved ability of a cell or a population of cells to survive under conditions of oxidative stress, for example, in the presence of reactive oxygen species (ROS) as compared to a reference cell or population of cells.

“Host cell” refers to a cell that can be or has been engineered to produce a protein of interest. In one aspect, the host cell is transfected with an isolated polynucleotide sequence encoding a protein of interest. In one aspect, the protein of interest is a therapeutic protein. In one aspect, the protein of interest is an antibody, for example, a monoclonal antibody or an antigen-binding antibody fragment. In one aspect, the protein of interest is a bispecific antibody. “Host cell” refers not only to the cell into which a recombinant expression vector has been introduced, but also includes the progeny of such a cell. Because of modifications that may occur in succeeding generations, for example, due to environmental influences, such progeny may not be identical to the parent cell, but are still included within the scope of the term “host cell”. As used herein, the term “host cell” includes a population of host cells or a cell line. Host cells can include mammalian host cells, including, but not limited to, Chinese Hamster Ovary (CHO), baby hamster kidney (BHK), murine myeloma (NSO or SP2), or rat myeloma (YB2/0) cells. In one aspect, the host cell includes Chinese hamster ovary (CHO) cells.

A “hydrogen peroxide (H₂O₂)-evolved host cell” refers to a host cell that has been cultured in the presence of hydrogen peroxide that is characterized by an improved cell culture performance as compared to a non-evolved or parent host cell when cultured under the same culture conditions. In one aspect, the culture conditions include conditions of oxidative stress, for example, due to the presence of hydrogen peroxide or reactive oxygen species (ROS). Improved performance can be determined by measuring one or more cell performance parameters. “Cell performance parameters” refers to any parameter than can be measured that is indicative of cell viability, cell growth and/or productivity. Examples of cell performance parameters include, but are not limited to, viability, viable cell density, lactate levels in spent media, protein titer, specific productivity (qP), or a combination thereof. In one aspect, the improved performance results in a statistically significant (p<0.05) improvement in one or more cell performance parameters. Whether a change in a cell performance parameter is statistically significant can be determined using an appropriate t-test or other statistical test known to those of skill in the art.

A “parent” or “parental control” refers to a non-evolved host cell that has not been cultured in the presence of hydrogen peroxide to improve cell culture performance. In one aspect, the parent or parental control is a non-evolved mammalian host cell, such as a Chinese Hamster Ovary (CHO), baby hamster kidney (BHK), murine myeloma (NSO or SP2), or rat myeloma (YB2/0) cell. In one aspect, the parent or parental control is a non-evolved Chinese hamster ovary (CHO) cell.

“Viability” refers to a measure of the number of cells that are alive and capable of growth. Assays for determining cell viability are well-known in the art and include, for example, assays for markers that are indicators of metabolically active cells, such as ATP levels, the ability to reduce a substrate and enzymatic or protease activities unique to living cells. In one aspect, cell viability is calculated by determining the total cell count minus the count of nonviable or dead cells. In one aspect, cell viability can be determined using a commercially-available automated cell culture analysis system, including, for example, Vi-Cell XR Cell Viability Analyser (Beckman Coulter, USA).

“Improved” or “increased” viability refers to a state in which a population of cells, for example, a population of H₂O₂-evolved cells, exhibits an increased number of viable cells relative to a reference population, for example, a population of non-evolved or parental control cells, under similar culture conditions.

“Cell density” refers to the number of cells per unit volume of culture media. “Viable cell density” or VCD refers to the number of live cells per unit volume of cell culture media under a set of culture conditions. Assays for determining viable cell density are known and include, for example, trypan blue dye exclusion. In one aspect, viable cell density is determined using a commercially-available automated system. Improved viable cell density, refers to a state in which a population of cells, for example, a population of H₂O₂-evolved cells, exhibits increased numbers of viable cells relative to a reference population, for example, a population of non-evolved or parental control cells, under similar culture conditions.

Lactate is a by-product produced during mammalian cell culture. High lactate levels can be caused by high aerobic glycolysis, known as the Warburg effect, and are often associated with impaired cell culture performance. Accumulation of lactate can impact the buffering capacity of the cell culture media, which can result in a decrease in pH. “Lactate accumulation” can be a limiting factor during a cell culture process, especially in high cell density cell cultures. Reducing lactate accumulation during cell culture can improve growth, productivity, and robustness of the cell culture process.

“Titer” refers to the volumetric concentration of a product, for example, a recombinantly produced protein of interest, in a solution, for example, cell culture media and can be expressed as mg/L or g/L. As used herein, “increased titer” refers to a titer that is at least about 1.5-fold higher than the titer of a reference cell line, for example, a parental cell line. In one aspect, the H₂O₂-evolved cell line has a titer that is from about 1.5-fold to about 3.5-fold as compared to a non-evolved parental control.

“Specific productivity” or “qP” refers to the productivity of a cell line producing a heterologous protein in cell culture and refers to the product expression rate, for example, per cell, or per measure of cell mass or volume. Specific productivity can be calculated by dividing the final yield by the viable cells, and can be expressed in units of pg/cell/day. In one aspect, specific productivity refers to the productivity of a cell line producing a heterologous protein. In one aspect, specific productivity refers to the productivity of a cell line producing a heterologous a bispecific antibody,

“Expression” refers to transcription and translation of a gene within a host cell. The level of expression can be determined based on the amount of corresponding mRNA that is present in the cell or the amount of protein encoded by the gene that is produced by the cell. Methods for detecting mRNA or protein are known. For example, mRNA can be detected by northern blot hybridization and protein encoded by a gene can be detected by assaying for a biological activity using a Western blot or radioimmunoassay.

“Increased expression” refers to an expression level of a particular gene sequence in a cell or organism that is increased relative to a control cell or organism. In one aspect, the increased level of gene expression is statistically significant (p<0.05). Whether an increase in expression relative to a control is statistically significant can be determined using an appropriate t-test or other statistical test known to those of skill in the art.

“Decreased expression” refers to a reduced expression level of a particular gene sequence in a cell or organism that is decreased relative to a control cell or organism. In one aspect, the decreased level of gene expression is statistically significant (p<0.05). Whether a decrease in expression relative to a control is statistically significant can be determined using an appropriate t-test or other statistical test known to those of skill in the art.

“Cell culture” refers to a population of cells cultivated under conditions suitable for cell growth, maintenance, differentiation, transfection, or propagation. “Cell culture” can also refer to a population of cells and the cell culture media in which they are maintained. In one aspect, the term “cell culture” refers a population of host cells capable of producing a recombinant protein of interest. An adherent culture refers to a culture in which the cells are grown as monolayers on an artificial substrate. A suspension culture refers to a culture in which the cells are free-floating in a culture medium. In one aspect, host cells are grown in an adherent cell culture. In one aspect, host cells are grown in a suspension cell culture. In one aspect, H₂O₂-evolved host cells are grown in a suspension cell culture. Cell culture processes are known and include, for example, batch, fed-batch or perfusion processes.

“Culture media” refers to a solution that provides nutrients, for example, vitamins, amino acids, essential nutrients, and salts, and conditions, for example, buffering, to maintain living cells and support their growth and propagation. Typically, culture media includes one or more of the following components: an energy source, for example, a carbohydrate such as glucose; amino acids, for example, the basic twenty amino acids plus cysteine; vitamins and/or other organic compounds; free fatty acids; and trace elements. The nutrient solution may optionally be supplemented with one or more additional components, including, but not limited to: hormones or growth factors; salts and buffers; nucleosides and bases; protein or tissue hydrolysates; or combinations thereof.

“Conditioned culture media” refers to culture media that has been incubated with cultured cells and includes metabolites, growth factors and proteins secreted into the medium by the cultured cells.

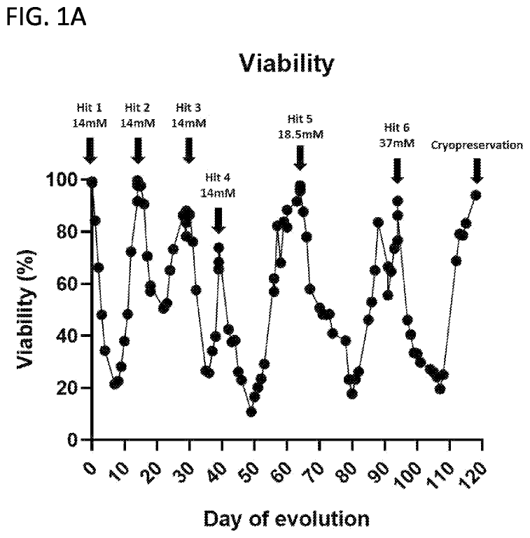

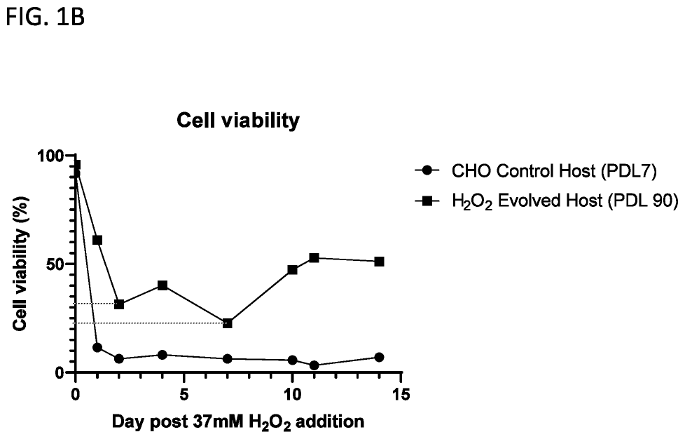

Population doubling (PDL) is a measure of the age of a particular culture of a cell line and refers to the total number of times the cells in the population have doubled. In one aspect, PDL refers to the number of times cells from a master cell bank have doubled. In one aspect, the cells in the master cell bank are H₂O₂-evolved cells as described herein. In one aspect, the cells in the master cell bank includes cells that were evolved following the process shown schematically in FIG. 10. In one aspect, the cells in the master cell bank are cells that were evolved following the process shown in FIG. 1A.

Passage number refers to the number of times cells in a culture have been subcultured.

“Batch” cultivation is a discontinuous process, in which a fixed volume of culture medium is inoculated with the host cells and no additional growth medium is added. Typically, the only material added and removed during a batch culture process is air/gas exchange, antifoam and pH controlling agents. Often, the batch cultures are shaken or stirred to maintain a desired degree of homogeneity and to improve oxygen transfer The number of cells increase, usually exponentially, until a maximum is reached, after which growth is arrested and the cells die. To recover product, cells are removed from the medium. Generally, batch culture proceeds in a fixed volume, for a fixed duration, with a single harvest in which the cells die or are discarded at the end of the process.

“Fed-batch” cultivation is a variation on batch culture in which a feed is added either periodically or continuously during the process. Fed batch culture usually proceeds in a substantially fixed volume, for a fixed duration, and with a single harvest either when the cells have died or at an earlier, predetermined point.

In a “perfusion” culture, culture medium is perfused through the cell culture at a high rate while cells are retained or recycled back into the reactor by sedimentation, centrifugation or filtration. In a perfusion culture, the perfusing medium helps remove metabolites, such as lactate, from the culture medium.

“Transfect” or “transfection” refers to a method in which a construct that includes heterologous polynucleotide is introduced into a host cell to generate a genetically modified or transgenic cell. Transfection methods are known and include, but are not limited to, liposome-mediated transfection, calcium phosphate co-precipitation, electroporation, polycation (such as DEAE-dextran)-mediated transfection, protoplast fusion, viral infections and microinjection. Transfection can be transient or stable. In transient transfection, the construct containing heterologous polynucleotide is not integrated into the genome of the host cell. In stable transfection, the construct containing the heterologous polynucleotide is integrated into the genome of the host cell, for example, the construct can be integrated into a nuclear or organelle genomes, or located episomally wherein the polynucleotide is maintained within the genome of the host cell and passed on to future generations.

The terms “nucleic acid”, “polynucleotide” and “oligonucleotide” can be used interchangeably to refer to a polymeric compound that includes covalently linked subunits of nucleotides. Polynucleotides can be single or double stranded. Polynucleotides include, but are not limited to, polyribonucleic acid (RNA) and polydeoxyribonucleic acid (DNA). DNA includes, but is not limited to, cDNA, genomic DNA, and synthetic DNA. RNA includes, but is not limited to, transfer RNA (tRNA), ribosomal RNA (rRNA), and messenger RNA (mRNA).

The terms “polypeptide”, “protein” and “peptide” are used interchangeably herein to refer to a polymer of amino acids of any length and can refer to a complex that includes a plurality of polypeptide chains. The amino acid polymer can be straight, branched or cyclic. An amino acid may be a naturally-occurring or non-naturally-occurring amino acid, or a variant amino acid. A protein can be naturally occurring or recombinantly produced. A protein can be secreted, membrane bound, or intracellular. Proteins can include therapeutic proteins or other proteins of commercial interest.

“Isolated” refers to a polynucleotide, polypeptide or cell that has been separated and/or removed from a component of its natural environment. For example, an isolated cell can be removed from an animal and placed in a culture dish or another animal. Isolated does not mean that the cell is removed from all other cells. A group of cells can also be isolated. A polynucleotide or polypeptide is isolated when it is removed from cellular material typically associated with the polynucleotide or polypeptide, or substantially free of chemical precursors or other chemicals when chemically synthesized. An “isolated polynucleotide” or “isolated polypeptide” can refer to a substantially purified polynucleotide or polypeptide, respectively. In one aspect, the isolated polynucleotide or polypeptide is present in a non-native environment, for example, a heterologous cell, tissue, or animal.

“Heterologous” refers to a polynucleotide or polypeptide that does not naturally occur in an organism that has been introduced, for example, using recombinant molecular biology techniques. A heterologous polynucleotide or polypeptide can include a polynucleotide or polypeptide that is the same as or different than a polynucleotide or polypeptide that naturally occurs in the organism. For example, “heterologous polynucleotide” can refer to a gene introduced into a cell via recombinant methods, for example, introduced on a plasmid and “heterologous polypeptide” can refer to a polypeptide not naturally synthesized by a an organism that is expressed from a polynucleotide that is introduced by recombinant molecular biology techniques. “Recombinant” when used with reference to a cell, polynucleotide, polypeptide, or vector, indicates that the cell, polynucleotide, polypeptide or vector, has been modified by the introduction of a heterologous polynucleotide or polypeptide or by alteration of a native polynucleotide or polypeptide, or a cell derived from a cell so modified.

A “protein of interest” includes proteins, polypeptides, fragments, and peptides, which can be expressed by a host cell. Proteins of interest include, for example, antibodies, enzymes, cytokines, lymphokines, adhesion molecules, receptors and derivatives or fragments thereof. In one aspect, the protein of interest is an antibody or an antigen-binding antibody fragment. In one aspect, the protein of interest is a bispecific antibody.

“Antibodies” and “immunoglobulins” can be used interchangeably and include, but are not limited to, monoclonal antibodies (including full-length monoclonal antibodies), polyclonal antibodies, multispecific antibodies, bispecific antibodies, human antibodies, humanized antibodies, camelised antibodies, single-chain Fvs (scFv), single-chain antibodies, single domain antibodies, domain antibodies, Fab fragments, F(ab′)2 fragments, disulfide-linked Fvs (dsFv), anti-idiotypic (anti-Id) antibodies, intrabodies, and antigen-binding fragments thereof. Antibodies can also include peptide fusions with antibodies or portions thereof such as a protein fused to an Fc domain. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

Naturally occurring IgG antibodies typically include four polypeptide chains: two heavy (H) chains and two light (L) chains that are inter-connected by disulfide bonds. Each heavy chain includes a heavy chain variable region (VH) and a heavy chain constant region, which includes three domains, CH1, CH2 and CH3. Each light chain includes a light chain variable region (VL) and a light chain constant region, which includes one domain, CL. The VH and VL regions can be further subdivided into regions of hypervariability, termed complementarity determining regions (CDR), interspersed with regions that are more conserved, termed framework regions (FR). Each VH and VL includes three CDRs and four FRs. Heavy chain CDRs can be abbreviated as HCDR1, HCDR2 and HCDR3 and light chain CDRs can be abbreviated as LCDR1, LCDR2 and LCDR3.

The term “monoclonal antibody” refers to an antibody obtained from a population of substantially homogeneous antibodies. Monoclonal antibodies are directed against a single epitope, in contrast to polyclonal antibody preparations which typically include antibodies directed against different epitopes. The term “monoclonal” indicates the character of the antibody obtained from a substantially homogeneous population of antibodies, and does not require engineering of the antibody by any particular method.

In one aspect, the antibody is a multispecific or bispecific antibody. As use herein, a “multispecific” or “bispecific” antibody is an antibody that is capable of selectively binding two or more epitopes. In one aspect, the antibody is a bispecific antibody having two different heavy chain variable domains, with each heavy chain variable domain specifically binding a different epitope. In one aspect, the antibody is a bispecific antibody having two different heavy chain variable domains and two different light chain variable domains. The epitopes recognized by the bispecific antibody can be on the same or a different target, for example, the epitopes can be on the same or different protein. In one aspect, the bispecific antibody recognizes different epitopes on the same antigen. In one aspect, the bispecific antibody recognizes different epitopes on different antigens.

“Difficult to express” or “DTE” refers to a protein for which recombinant production is difficult, for example, proteins that are difficult to express at titers sufficient for clinical or therapeutic use. For example, production of a DTE protein can be difficult because the protein is prone to mis-folding, degradation or aggregation. Sometimes, a protein can be difficult to express because it is cytotoxic. Proteins that include multiple polypeptide chains, such as antibodies, can sometimes be difficult to express. In particular, bispecific antibodies can be difficult to express. Difficult to express proteins can be associated with increased levels of oxidative stress. For example, a difficult to express protein may result in the generation of reactive oxygen species as by-products of the protein folding process that occurs in the endoplasmic reticulum (ER). The increased levels of oxidative stress can lead to an impaired reduction-oxidation (redox) balance, resulting in oxidative stress.

The phrase “pharmaceutically acceptable” means approved by a regulatory agency of a Federal or a state government, or listed in the U.S. Pharmacopeia, European Pharmacopia or other generally recognized pharmacopeia for use in animals, for example, for use in humans.

### B. Overview

Provided herein are host cells for producing a protein of interest and methods of making and using the same. In one aspect, the host cell or cell line is obtained using directed evolution to improve performance and to enhance productivity for producing of a protein of interest. In one aspect, improved performance includes one or more performance parameters such as increased viable cell density, increased viability, decreased lactate levels, increased titer, and increased specific productivity (qP). In one aspect, the improved performance results in a statistically significant (p<0.05) improvement in one or more cell performance parameters. Whether a change in a cell performance parameter is statistically significant can be determined using an appropriate t-test or other statistical test known to those of skill in the art.

In one aspect, directed evolution is used to generate a host cell line with improved performance by culturing the host cells in the presence of hydrogen peroxide (H₂O₂) to generate a H₂O₂-evolved host cell or cell line. In one aspect, a H₂O₂-evolved host cell is provided. In one aspect, methods of making and using a H₂O₂-evolved host cell are provided. In one aspect, the host cell has improved performance under conditions of cellular stress. In one aspect, cellular stress includes transfection, for example stable transfection. In one aspect, cellular stress includes recombinant expression of a heterologous protein. In one aspect, cellular stress includes expression of a difficult to express protein. In one aspect, cellular stress includes expression of an antibody. In one aspect, cellular stress includes expression of a bispecific antibody. In one aspect, cellular stress includes cell culture processes such as a batch or fed-batch cell culture process. In one aspect, the host cell is resistant to oxidative stress. In one aspect, the cell line is a mammalian cell line. In one aspect, the cell line is a Chinese Hamster Ovary (CHO) cell line. In one aspect, the cell line is a suspension adapted CHO cell line.

In one aspect, the host cell is a mammalian host cell with improved performance when expressing a protein of interest. In one aspect, the host cell or cell line is a mammalian host cell with improved performance for producing an antibody or an antigen-binding antibody fragment. In one aspect, the host cell or cell line is a mammalian host cell with improved performance for producing a bispecific antibody.

In one aspect, the host cell is a mammalian host cell with improved performance when expressing a protein of interest that is difficult to express (DTE). In one aspect, the DTE protein is a bispecific antibody.

The manufacture of bispecific antibodies is often hindered by lower product yields as compared to yields obtained for monoclonal antibodies. It has been shown that reactive oxygen species (ROS) can negatively impact antibody production in cell culture and that strategies that boost cellular antioxidant capacity appear to be beneficial for protein expression. While not wishing to be bound by theory, it is believed that intracellular production of bispecific antibodies may induce oxidative stress. In one aspect, an evolved host cell is provided that expresses bispecific antibodies with a higher product yield as compared to a non-evolved parent host cell. In one aspect, the evolved host cell expresses a protein of interest at a statistically significant (p<0.05) higher titer than a non-evolved host cell. Whether a change in expression is statistically significant can be determined using an appropriate t-test or other statistical test known to those of skill in the art.

Provided herein is an evolved host cell generated using directed evolution that has heritable resistance to hydrogen peroxide over many generations. The H₂O₂-evolved host cell displays enhanced antioxidant capacity due to an increase in antioxidants such as glutathione (GSH) and the upregulation of several, diverse antioxidant defense genes, including those involved in glutathione (GSH) biosynthesis and turnover, as well as H₂O₂ elimination. In one aspect, the evolved host cell has a statistically significant (p<0.05) increased expression of one or more antioxidant defense genes than a non-evolved host cell. Whether an increase in expression is statistically significant can be determined using an appropriate t-test or other statistical test known to those of skill in the art.

The global changes in antioxidant pathways confers resistance to a diverse subset of oxidative stressors. However, it is not necessary that the cells be under oxidative stress to observed the improved performance. Additionally, the H₂O₂-evolved host cell has superior transfection recovery times, demonstrates improved growth and viability in a fed batch production process, and has elevated expression levels for difficult to express proteins such as bispecific antibodies as compared to unevolved CHO control cells.

### C. Reactive Oxygen Species (ROS)

Reactive oxygen species (ROS) are a group of molecules derived from oxygen, which, due to their oxygen content or the presence of unpaired electrons, display high reactivity towards a large array of biomolecules. The metabolic adaptations required to support high-level expression of recombinant proteins can result in an increase in the generation of ROS. For example, mammalian cell culture used in the production of recombinant proteins require an elevated level of protein folding and secretion. This increased activity within the Endoplasmic Reticulum can result in an increase in the production of ROS. Additionally, media components can react with oxygen, light, and other components to generate ROS. (Chevallier et al. (2020) “Oxidative stress-alleviating strategies to improve recombinant protein production in CHO cells.” Biotechnol. Bioeng. 117(4):1172-1186).

In one aspect, a host cell is provided that has an increased resistance to oxidative stress when compared to a control. In one aspect, a mammalian host cell capable expressing a protein of interest is provided that has an increased resistance to oxidative stress when compared to a parental control. In one aspect, a hydrogen peroxide (H₂O₂)-evolved mammalian host cell capable expressing a protein of interest is provided that has an increased resistance to oxidative stress when compared to a parental control.

As used herein, “resistance to oxidative stress” refers to the ability of a cell to survive in the presence of oxidative stress. In one aspect, “resistance to oxidative stress” refers to the ability of a cell to survive in the presence of reactive oxygen species (ROS). In one aspect, “resistance to oxidative stress” refers to the ability of a cell to survive in the presence of increased levels of reactive oxygen species (ROS). In one aspect, “resistance to oxidative stress” refers to a cell with improved performance during cell culture. In one aspect, the improved performance during cell culture is observed, even when an increase in ROS levels is not observed. In one aspect, “resistance to oxidative stress” refers to a cell with improved performance in fed-batch cell culture. In one aspect, “resistance to oxidative stress” refers to a cell with improved production yields during cell culture.

In one aspect, resistance to oxidative stress is associated with changes in gene expression patterns, for example, changes in expression of genes involved in the cells antioxidant defense system. In one aspect, resistance to oxidative stress refers to the ability of a transfected cell to efficiently produce a heterologous protein of interest during cell culture. In one aspect, resistance to oxidative stress refers to the ability of a transfected cell to efficiently produce high titers of a heterologous protein of interest during cell culture. In one aspect, resistance to oxidative stress refers to the ability of a transfected cell to efficiently produce high titers of a difficult to express heterologous protein during cell culture. In one aspect, resistance to oxidative stress refers to the ability of a transfected mammalian cell to efficiently produce high titers of a heterologous antibody during cell culture. In one aspect, resistance to oxidative stress refers to the ability of a transfected mammalian cell to efficiently produce high titers of a heterologous bispecific antibody during cell culture. In one aspect, resistance to oxidative stress refers to the ability of a transfected Chinese hamster ovary (CHO) cell to efficiently produce high titers of a heterologous antibody during cell culture. In one aspect, resistance to oxidative stress refers to the ability of a transfected Chinese hamster ovary (CHO) cell to efficiently produce high titers of a heterologous antibody during a fed-batch cell culture. In one aspect, resistance to oxidative stress refers to the ability of a transfected Chinese hamster ovary (CHO) cell to efficiently produce high titers of a heterologous bispecific antibody during cell culture. In one aspect, resistance to oxidative stress refers to the ability of a transfected Chinese hamster ovary (CHO) cell to efficiently produce high titers of a heterologous bispecific antibody during a fed-batch cell culture. As used herein, high titer refers to a titer in the grams per liter (g/L) scale. In one aspect, “high titer” refers to a titer that is a statistically significant (p<0.05) increase over the titer observed from a non-evolved host cell. Whether a change in titer is statistically significant can be determined using an appropriate t-test or other statistical test known to those of skill in the art. In one aspect, “high titer” refers to a titer that is at least about 1.5 fold higher than a titer of a parent control.

### D. Antioxidant Defense Systems

To counterbalance the effect of reactive oxidant species (ROS), including, for example, ROS formed as a by-product of normal cell activity, mammalian cells have developed an array of “antioxidant defense systems” that include a variety enzymatic and non-enzymatic defense mechanisms to prevent intracellular damage by ROS. (Chevallier et al. (2020) “Oxidative stress-alleviating strategies to improve recombinant protein production in CHO cells.” Biotechnol. Bioeng. 117(4):1172-1186). Antioxidant defense systems can be divided into two (2) categories: enzymatic and non-enzymatic antioxidants.

Enzymatic antioxidants include, but are not limited to, superoxide dismutase (SOD), catalase and glutathione peroxidase (GPx-1), which work together to catalytically remove reactive oxygen species: SOD converts superoxide radical (O₂⁻·) into hydrogen peroxide and molecular oxygen; and catalase and glutathione peroxidase (GPx-1) break down hydrogen peroxide to form oxygen and water.

Non-enzymatic antioxidants include, but are not limited to, low molecular weight compounds such as vitamin C, vitamin E and glutathione (GSH). (Valko et al. (2007) “Free radicals and antioxidants in normal physiological functions and human disease”. Int J Biochem Cell Biol. 39(1):44-84). Glutathione is a tripeptide of cysteine, glycine and glutamic acid (γ-L-glutamyl-L-cysteinyl glycine) that is present in all cell types at millimolar concentrations and acts as a major redox buffer effecting a broad range of intracellular systems (Forman et al. (2009) “Glutathione: overview of its protective roles, measurement, and biosynthesis”. Mol Aspects Med. 30(1-2):1-12).

Glutathione exists in a reduced form (GSH) and an oxidized form, glutathione disulfide (GSSG). (Pizzorno, J. (2014) “Glutathione!” Integr. Med (Encinitas). 13(1):8-12). As used herein, total glutathione refers to the sum of GSH and GSSG found in a cell. The ratio of total glutathione to glutathione disulfide (GSH:GSSG) determines cell redox status of cells and can be an indicator of oxidative stress.

Depletion of the reduced form of GSH has been linked to decreased specific productivity (qP) of manufacturing cell lines (Handlogten et al. (2020) “Online Control of Cell Culture Redox Potential Prevents Antibody Interchain Disulfide Bond Reduction”. Biotechnol Bioeng. 117(5):1329-1336) and proteomic work by Orellana et al demonstrated that high antibody producing CHO cell lines up-regulated GSH biosynthetic pathways (Orellana et al. (2015) “High-antibody-producing Chinese hamster ovary cells up-regulate intracellular protein transport and glutathione synthesis”. J Proteome Res. 14(2):609-18). These data are supported by observations that high producer cell lines had increased cellular GSH content (Chong et al. (2012) “LC-MS-based metabolic characterization of high monoclonal antibody-producing Chinese hamster ovary cells”. Biotechnol Bioeng. 109(12):3103-11). Consistently, the modulation of GSH synthetic enzymes, through targeted genetic overexpression, has been shown to improve mAb titers (Orellana et al. (2017) “Overexpression of the regulatory subunit of glutamate-cysteine ligase enhances monoclonal antibody production in CHO cells”. Biotechnol Bioeng. 114(8):1825-36).

GSH is synthesized from constituent amino acids in two steps. In the first step, γ-glutamylcysteine is formed from glutamate and cysteine; in the second step, glutathione (GSH) is formed from γ-glutamylcysteine and glycine. The first step of GSH biosynthesis is catalyzed by glutamate cysteine ligase (GCL), a heterodimer that includes a heavy or catalytic subunit (GCLC) and a light or modifier subunit (GCLM). The GCLC subunit exhibits all of the catalytic activity of the enzyme and GCLM is enzymatically inactive. Glutathione synthetase (GS) is a homodimeric enzyme that catalyzes the ligation of γ-glutamylcysteine and glycine to generate glutathione (GSH).

GSH donates its electron to hydrogen peroxide (H₂O₂) to reduce H₂O₂ to oxygen (O₂) and water (H₂O) generating oxidized glutathione disulfide (GSSG) in the process. This reaction is catalyzed by glutathione peroxidase (GPx-1). GSSG is then reduced to form GSH by glutathione reductase. Increased levels of oxidative stress, result in an accumulation of intracellular GSSG, thereby reducing the total glutathione:GSSG (GSH:GSSG) ratio. Cells exposed to oxidant stress generally have a total glutathione:GSSG (GSH:GSSG) ratio between about 1 and about 10. Accumulation of GSSG due to oxidative stress can be toxic to cells.

The antiporter system x_{c}⁻ imports the amino acid cystine (the oxidized form of cysteine) into cells with a 1:1 counter-transport of glutamate. Cysteine is a rate-limiting substrate for glutathione (GSH) and, along with cystine, forms a redox couple. (Lewernz et al. (2013) “The cystine/glutamate antiporter system x_{c}⁻ in health and disease: from molecular mechanisms to novel therapeutic opportunities.” Antioxid. Redox Signal. 18(5):522-555). As used herein, xCT refers to the gene encoding the cystine/glutamate antiporter.

Other antioxidants include thioredoxin reductase 1 and peroxiredoxin 6, which are linked to improved recombinant protein expression (Kelly, et al. (2015) “Re-programming CHO cell metabolism using miR-23 tips the balance towards a highly productive phenotype”. Biotechnol J 10(7):1029-40). The transcription factor Forkhead BoxA1 (Foxa1) has also been linked to improved expression of difficult to express (DTE) antibodies through a mechanism involving reduced oxidative stress (Berger et al. (2020) “Overexpression of transcription factor Foxa1 and target genes remediate therapeutic protein production bottlenecks in Chinese hamster ovary cells”. Biotechnol Bioeng. 117(4):1101-16). While not wishing to be bound by theory, it is believed that upregulation of antioxidants is beneficial for the expression of recombinant proteins in mammalian host cells, such as CHO cells.

In one aspect, a host cell is provided in which in which a level of one or more components of an antioxidant defense system are increased when compared to a parental control. In one aspect, the level of one or more components of an antioxidant defense system are increased when the host cell is expressing a heterologous protein. However, the level of one or more components of an antioxidant defense system can also be increased when the host cell is not expressing a heterologous protein. In one aspect, a H₂O₂-evolved host cell is provided in which a level of one or more components of an antioxidant defense system are increased when compared to a parental control. In one aspect, a H₂O₂-evolved CHO host cell is provided in which a level of one or more components of an antioxidant defense system are increased when compared to a parental control. In one aspect, components of the antioxidant defense system include, but are not limited to, glutathione (GSH), oxidized glutathione (GSSG), glutathione synthetase (GSS); glutamate cysteine ligase modifier subunit (GCLM); catalase; cysteine/glutamate antiporter light chain (xCT); glutathione peroxidase-1 (GPx-1). In one aspect, a host cell is provided in which in which a level of one or more components of an antioxidant defense system that include, but are not limited to, glutathione (GSH), oxidized glutathione (GSSG), glutathione synthetase (GSS); glutamate cysteine ligase modifier subunit (GCLM); catalase; cysteine/glutamate antiporter light chain (xCT); glutathione peroxidase-1 (GPx-1), and combinations thereof, are increased when compared to a parental control. In one aspect, the increase in the level of one or more components of antioxidant defense system is statistically significant (p<0.05) as compared to the level in a non-evolved control. Whether an increase in the level of a component of an antioxidant defense system is statistically significant can be determined using an appropriate t-test or other statistical test known to those of skill in the art.

In one aspect, the H₂O₂-evolved host cell has an increased level of GSH when compared to the parental control. In one aspect, the H₂O₂-evolved host cell has from about a 1% to about 25%, higher level of GSH than the parental control. In one aspect, the H₂O₂-evolved host cell has from about a 2% to about 20% higher level of GSH than the parental control. In one aspect, the H₂O₂-evolved host cell has from about a 3% to about 10% higher level of GSH than the parental control. In one aspect, the H₂O₂-evolved host cell has at least about 1%, about 2%, about 3%, about 4%, about 5%, about 6%, about 7%, about 8%, about 9%, about 10%, about 15%, or about 20% higher level of GSH than the parental control.

In one aspect, the H₂O₂-evolved host cell has an increased ratio of total glutathione to oxidized glutathione (GSSG) (GSH:GSSG) when compared to a parental control. In one aspect, the ratio of total glutathione to GSSG (GSH:GSSG) is increased by about 1% to about 15% as compared to a parental control. In one aspect, the ratio of total glutathione to GSSG (GSH:GSSG) is increased by about 2% to about 10% as compared to a parental control. In one aspect, the ratio of total glutathione to GSSG is increased by at least about 1%, about 2%, about 3%, about 4%, about 5%, about 6%, about 7%, about 8%, about 9%, about 10%, or about 15% as compared to the parental control. In one aspect, the ratio of total glutathione to GSSG (GSH:GSSG) is from about 2.5:1 to about 3:1. In one aspect, the ratio of total glutathione to GSSG (GSH:GSSG) is at least about 2.5:1, about 2.6:1, about 2.7:1, about 2.8:1, about 2.9:1 or about 3:1.

In one aspect, one or more antioxidant defense genes of the H₂O₂-evolved host cell are upregulated as compared to a parental control. In one aspect, one or more antioxidant defense genes are selected from: glutathione synthetase (GSS); glutamate cysteine ligase modifier subunit (GCLM); catalase; cysteine/glutamate antiporter light chain (xCT); glutathione peroxidase-1 (GPx-1); and combinations thereof. In one aspect, the increase in expression of one or more antioxidant defense genes is statistically significant (p<0.05) as compared to a non-evolved control. Whether an increase in expression is statistically significant can be determined using an appropriate t-test or other statistical test known to those of skill in the art.

In one aspect, GSS expression is increased at least about 10%, about 20%, about 30%, about 40%, about 50%, about 60%, about 70%, about 80%, about 90%, about 100% and up to about 150%, about 200%, about 250% or about 300% as compared to a parental control. In one aspect, GSS expression is increased about 10% to about 300% as compared to the parental control. In one aspect, GSS expression is increased about 25% to about 200% as compared to the parental control.

In one aspect, GCLM expression is increased at least about 10%, about 15%, about 20%, about 25%, about 30%, about 35%, about 40%, about 45%, about 50%, about 55%, about 60%, about 65%, about 70%, or about 75% as compared to the parental control. In one aspect, GCLM expression is increased about 10% to about 100% as compared to a parental control. In one aspect, GCLM expression is increased about 25% to about 75% as compared to a parental control.

In one aspect, catalase expression is increased at least about 10%, about 15%, about 20%, about 25%, about 30%, about 35%, about 40%, about 45%, about 50%, about 55%, about 60%, about 65%, about 70%, or about 75% as compared to the parental control. In one aspect, catalase expression is increased about 10% to about 100% as compared to a parental control. In one aspect, catalase expression is increased about 25% to about 75% as compared to a parental control.

In one aspect, xCT expression is increased at least about 10%, about 15%, about 20%, about 25%, about 30%, about 35%, about 40%, about 45%, or about 50% as compared to the parental control. In one aspect, xCT expression is increased about 10% to about 50% as compared to the parental control.

In one aspect, GPx-1 expression is increased at least about 10%, about 15%, about 20%, about 25%, about 30%, about 35%, about 40%, about 45%, or about 50% as compared to the parental control. In one aspect, GPx-1 expression is increased about 10% to about 100% as compared to the parental control. In one aspect, GPx-1 expression is increased about 10% to about 50% as compared to the parental control.

### E. Performance Parameters

In one aspect, a host cell is provided that has improved performance as compared to a control cell. In one aspect, a population of host cells is provided that has improved performance as compared to a population of control cells. In one aspect, a population of evolved host cells is provided that has improved performance as compared to a population of parental control cells. In one aspect, a population of H₂O₂-evolved cells is provided that has improved performance as compared to a population of parental control cells. In one aspect, a population of H₂O₂-evolved CHO cells is provided that has improved performance as compared to a population of parental control CHO cells.

In one aspect, improved performance is demonstrated by an improvement in one or more performance parameters that include, but are not limited to, viable cell density, viability, lactate levels, titer, specific productivity (qP) or combinations thereof. In one aspect, improved performance is demonstrated by one or more of the following: increased viable cell density, increased viability, decreased lactate levels, increased titer, increased specific productivity (qP), or a combination thereof. In one aspect, the improved performance is reflected in a statistically significant (p<0.05) change in one or more performance parameters as compared to a non-evolved control. Whether a change in a performance parameter is statistically significant can be determined using an appropriate t-test or other statistical test known to those of skill in the art.

In one aspect, a host cell is provided that has improved performance as compared to a control cell when cultured in a batch, fed-batch, perfusion or continuous cell culture process. In one aspect, a host cell is provided that has improved performance as compared to a control cell when cultured in a batch cell culture process. In one aspect, a host cell is provided that has improved performance as compared to a control cell when cultured in a fed-batch cell culture process. In one aspect, a host cell is provided that has improved performance as compared to a control cell when cultured in a perfusion cell culture process. In one aspect, a host cell is provided that has improved performance as compared to a control cell when cultured in a continuous cell culture process.

In one aspect, a mammalian host cell is provided that has improved performance as compared to a control cell when cultured in a fed-batch cell culture process. In one aspect, a CHO cell is provided that has improved performance as compared to a control cell when cultured in a fed-batch cell culture process. In one aspect, an evolved host cell is provided that has improved performance as compared to a parental control cell when cultured in a fed-batch cell culture process. In one aspect, an H₂O₂-evolved host cell is provided that has improved performance as compared to a non-evolved parental control cell when cultured in a fed-batch cell culture process. In one aspect, an H₂O₂-evolved CHO host cell is provided that has improved performance as compared to a non-evolved parental control cell when cultured in a fed-batch cell culture process.

In one aspect, an H₂O₂-evolved cell is provided that has improved performance as compared to a population of parental control cells when expressing a protein of interest. In one aspect, the protein of interest expressed by the H₂O₂-evolved cell is a difficult to express protein. In one aspect, the protein of interest expressed by the H₂O₂-evolved cell includes more than one polypeptide chain. In one aspect, the protein of interest expressed by the H₂O₂-evolved cell is an antibody or antigen-binding antibody fragment. In one aspect, the protein of interest expressed by the H₂O₂-evolved cell is a monoclonal antibody. In one aspect, the protein of interest expressed by the H₂O₂-evolved cell is a bispecific antibody.

In one aspect, the population of H₂O₂-evolved cells has an increased viable cell density as compared to a population of parental control cells. In one aspect, the population of H₂O₂-evolved cells has a peak viable cell density from about 15×10⁶ cells/mL to about 25×10⁶ cells/mL In one aspect, the population of H₂O₂-evolved cells has a peak viable cell density of at least about 15×10⁶ cells/mL, about 16×10⁶ cells/mL, about 17×10⁶ cells/mL, about 18×10⁶ cells/mL, or about 19×10⁶ cells/mL and up to about 20×10⁶ cells/mL.

In one aspect, the population of H₂O₂-evolved cells has increased viability as compared to a population of parental control cells. In one aspect, the population of H₂O₂-evolved cells has at least about 10%, about 20%, about 30%, about 40%, or about 50% increased viability compared to the population of parental control cells. In one aspect, the population of H₂O₂-evolved cells has at least about 10%, about 20%, about 30%, about 40%, or about 50% increased viability compared to the population of parental control cells when challenged with hydrogen peroxide. In one aspect, the population of H₂O₂-evolved cells has an increased viability compared to the population of parental control cells when challenged with at least about 5 mM, about 10 mM, about 15 mM, about 20 mM, about 25 mM, about 30 mM, about 35 mM, and up to about 40 mM hydrogen peroxide. In one aspect, the population of H₂O₂-evolved cells has an increased viability compared to a population of parental control cells when challenged with about 5 mM to about 40 mM hydrogen peroxide. In one aspect, the population of H₂O₂-evolved cells has an increased viability compared to the population of parental control cells when challenged with about 10 mM to about 40 mM hydrogen peroxide. In one aspect, the population of H₂O₂-evolved cells has an increased viability compared to the population of parental control cells when challenged with about 20 mM to about 40 mM hydrogen peroxide. In one aspect, the population of H₂O₂-evolved cells has an increased viability compared to the population of parental control cells when challenged with about 30 mM to about 40 mM hydrogen peroxide. In one aspect, the population of H₂O₂-evolved cells has an increased viability compared to the population of parental control cells when challenged with about 35 mM to about 40 mM hydrogen peroxide. In one aspect, the population of H₂O₂-evolved cells has an increased viability compared to the population of parental control cells when challenged with about 30 mM, about 31 mM, about 32 mM, about 33 mM, about 34 mM, about 35 mM, about 36 mM, about 37 mM, about 38 mM, about 39 mM, or about 40 mM hydrogen peroxide.

In one aspect, the population of H₂O₂-evolved cells produce a reduced level of one or more toxic metabolic by-products as compared to a population of parental control cells. In one aspect, the population of H₂O₂-evolved cells produce a reduced level of lactate as compared to a population of parental control cells. In one aspect, the population of H₂O₂-evolved cells generate a reduced level of lactate when expressing a heterologous protein as compared to a population of parental control cells when expressing the same heterologous protein. In one aspect, the population of H₂O₂-evolved cells exhibits a reduced lactate accumulation as compared to lactate accumulated by a reference population, for example, a non-evolved parental control. In one aspect, the reduced lactate accumulation for the population of H₂O₂-evolved cells is less than about 75%, about 70%, about 65%, about 60%, about 55%, about 50%, about 45%, about 40%, about 35%, about 30%, about 25% or about 20% of the lactate accumulation of a non-evolved parental control culture. In one aspect, reduced lactate accumulation is due to the H₂O₂-evolved cells releasing less lactate into the culture medium.

In one aspect, a population of H₂O₂-evolved cells is provided that express a protein of interest at a titer that is increased at least about 1.5 fold to 3.5 fold as compared to a population of parental control cells. In one aspect, a population of H₂O₂-evolved cells is provided that express a protein of interest at a titer that is at least about 1.5 fold, about 1.75 fold, about 2.0 fold, about 2.25 fold, about 2.5 fold, about 2.75 fold, about 3.0 fold, about 3.25 fold, or about 3.5 fold as compared to the population of parental control cells. In one aspect, the protein of interest is a difficult to express protein. In one aspect, the protein of interest includes more than one polypeptide chain. In one aspect, the protein of interest is an antibody or an antigen-binding antibody fragment. In one aspect, the protein of interest is a monoclonal antibody. In one aspect, the protein of interest is a bispecific antibody.

In one aspect, a population of H₂O₂-evolved cells expresses a protein of interest at a titer from about 0.50 g/L, about 0.55 g/L, about 0.60 g/L, about 0.65 g/L, about 0.70 g/L, about 0.75 g/L, about 0.80 g/L, about 0.85 g/L, about 0.90 g/L, about 0.95 g/L, and up to about 1.0 g/L, about 1.1 g/L, about 1.2 g/L, about 1.3 g/L, about 1.4 g/L, or about 1.5 g/L. In one aspect, a population of H₂O₂-evolved cells expresses a protein of interest at a titer from about 0.5 g/L to about 1.5 g/L. In one aspect, a population of H₂O₂-evolved cells expresses a protein of interest at a titer from about 0.5 g/L to about 1.1 g/L.

In one aspect, a population of H₂O₂-evolved cells expresses heterologous antibody at a titer from about 0.50 g/L, about 0.55 g/L, about 0.60 g/L, about 0.65 g/L, about 0.70 g/L, about 0.75 g/L, about 0.80 g/L, about 0.85 g/L, about 0.90 g/L, about 0.95 g/L, and up to about 1.0 g/L, about 1.1 g/L, about 1.2 g/L, about 1.3 g/L, about 1.4 g/L, or about 1.5 g/L. In one aspect, a population of H₂O₂-evolved cells expresses a heterologous antibody at a titer from about 0.5 g/L to about 1.5 g/L. In one aspect, a population of H₂O₂-evolved cells expresses an antibody at a titer from about 0.5 g/L to about 1.1 g/L.

In one aspect, a population of H₂O₂-evolved cells expresses a bispecific antibody at a titer from about 0.50 g/L, about 0.55 g/L, about 0.60 g/L, about 0.65 g/L, about 0.70 g/L, about 0.75 g/L, about 0.80 g/L, about 0.85 g/L, about 0.90 g/L, about 0.95 g/L, and up to about 1.0 g/L, about 1.1 g/L, about 1.2 g/L, about 1.3 g/L, about 1.4 g/L, or about 1.5 g/L. In one aspect, a population of H₂O₂-evolved cells expresses a bispecific antibody at a titer from about 0.5 g/L to about 1.5 g/L. In one aspect, the population of H₂O₂-evolved cells expresses a bispecific antibody at a titer from about 0.5 g/L to about 1.1 g/L. In one aspect, the titer of the bispecific antibody is at about 0.5 g/L, about 0.6 g/L, about 0.7 g/L, about 0.8 g/L, about 0.9 g/L, or about 1.0 g/L.

In one aspect, a population of H₂O₂-evolved cells is provided in which the cells have a specific productivity (qP) that is increased at least about 1.1 fold, about 1.2 fold, about 1.3 fold, about 1.4 fold, about 1.5 fold, about 1.6 fold, about 1.7 fold, about 1.8 fold, or about 1.9 fold, and up to about 2.0 fold as compared to a population of parental control cells.

In one aspect, the H₂O₂-evolved host cell has an improved viability under conditions of oxidative stress as compared to a parental control. In one aspect, the H₂O₂-evolved host cell has an improved viability after being cultured in the presence of a prooxidant chemical compound. As used herein, the term “prooxidant chemical compound” refers to a compound that induces oxidative stress, for example, by generating reactive oxygen species or by inhibiting one or more components of a cellular antioxidant defense system. Prooxidant chemical compounds are known, and include, but are not limited to, Menadione Sodium Bisulphite (MSB), Buthionine Sulfoximine (BSO), Mercaptosuccinic Acid (MS) and Cobalt chloride (CoCl). In one aspect, the H₂O₂-evolved host cell has an improved viability after about 24 hours to about 96 hours growth in the presence of about 5 μM to about 10 μM of a prooxidant chemical compound, or at least about 5 μM, about 6 μM, or about 7 μM, and up to about 8 μM, 0.9 μM, or 10 μM, of a prooxidant chemical compound. In one aspect, the H₂O₂-evolved host cell has an improved viability after at least about 24 hours, 36 hours or 48 hours and up to about 60 hours, about 72 hours, 84 hours or 96 hours growth in the presence of about 5 μM to about 10 μM of a prooxidant chemical compound.

### F. Directed Evolution

In one aspect, provided herein are methods of directing the evolution of a mammalian host cell and evolved host cells obtained by directed evolution. As used herein, “directed evolution” refers to a process for selecting a desired change in an organism in response to a condition of selective pressure. Advantageously, directed evolution provides a genome-wide approach for development of cells having a desired phenotype. In one aspect, a method is provided for evolving a host cell in the presence of hydrogen peroxide (H₂O₂).

In one aspect, a H₂O₂-evolved host cell is provided. As used herein, the term “H₂O₂-evolved host cell” includes host cells, cell lines and cell cultures that include the H₂O₂-evolved host cell. In one aspect, H₂O₂-evolved host cell expresses protein of interest. In one aspect, the H₂O₂-evolved host cell expresses a therapeutic protein. In one aspect, the H₂O₂-evolved host cell expresses an antibody or an antigen-binding antibody fragment. In one aspect, the H₂O₂-evolved host cell expresses a bispecific antibody. In one aspect, the host cell is a eukaryotic cell. In one aspect, the host cell is a mammalian host cell. In one aspect, the host cell is a human or rodent cell. In one aspect, the host cell is a mouse or hamster cell. Mammalian host cells include, but are not limited to sp20 cells, murine myeloma (NS0) cells, Chinese hamster ovary (CHO) cells, COS cells, 293 cells, PerC6™ cells, and hybridomas. In one aspect, the host cell is a CHO cell.

In one aspect, the H₂O₂-evolved host cell includes a heterologous gene encoding a protein of interest. In one aspect, the heterologous gene is stably integrated into the host cell genome. In one aspect, the heterologous gene is stably integrated into the host cell DNA. In one aspect, the heterologous gene is under the control of a constitutive promoter. In one aspect, the heterologous gene is under the control of an inducible promoter. In one aspect, the heterologous gene encodes a therapeutic protein of interest. In one aspect, the heterologous gene encodes a secreted protein.

In one aspect, the heterologous gene encodes an intracellular protein. In one aspect, the heterologous gene encodes a transmembrane protein. In one aspect, the heterologous gene encodes an antibody or an antigen-binding antibody fragment. In one aspect, an antibody or antigen-binding antibody fragment is constitutively expressed from the heterologous gene. In one aspect, the heterologous gene encodes a bispecific antibody. In one aspect, a bispecific antibody is constitutively expressed from the heterologous gene.

In one aspect, method of producing an evolved population of mammalian host cells is provided. In one aspect, the population of mammalian host cells has an increased resistance to oxidative stress. In one aspect, the method includes multiple rounds of culturing the population of cells in the presence of about 5 mM to about 20 mM, about 10 mM to about 15 mM, or about 15 mM to about 20 mM hydrogen peroxide (H₂O₂) and allowing the cells to recover until cells can survive in the presence of from about 20 mM to about 40 mM H₂O₂. In overview of the method is provided in FIG. 10. Briefly, the method includes:
(a) providing a population of cells;
(b) culturing the population of cells in a cell culture media;
(c) contacting the population of cells with about 5 mM to about 20 mM H₂O₂ to provide a population of transitional cells;
(d) resuspending the transitional cells in fresh cell culture media that does not include H₂O₂ and culturing until cells reach at least about 70% viability;
(e) repeating steps (c)-(d) to obtain a population of H₂O₂-evolved cells that can survive when contacted with about 20 mM to about 40 mM H₂O₂ and incubated for about 30 minutes to about 1 hour.

In one aspect, the method includes:
(a) providing a population of mammalian cells, for example, CHO cells;
(b) culturing the population of cells in a chemically defined cell culture media to at least about 90%, about 95%, about 96%, about 97%, about 98% or about 99% viability;
(c) contacting the population of cells with about 10 mM to about 20 mM H₂O₂ for about 30 minutes to about 2 hours to provide a population of primary transitional cells. In one aspect, the population of cells is contacted with about 10 mM to about 15 mM H₂O₂ for about 30 minutes to about 2 hours. In one aspect, the population of cells is contacted with about 13.5 mM, about 14 mM, or about 14.5 mM H₂O₂ for about 1 hour;
(d) resuspending the primary transitional cells in fresh chemically defined culture media that does not include H₂O₂ and culturing until at least about 60%, about 70% or about 80% viability;
(e) repeating steps (c)-(d) from about 3 times to about 5 times. In one aspect, the same concentration of H₂O₂ is used each time step (c) is repeated. In one aspect, the difference between the concentrations of H₂O₂ used each time step (c) is repeated is less than about 0.5 mM, less than about 1 mM or less than about 2 mM;
(f) contacting the population of primary transitional cells with about 15 mM to about 25 mM H₂O₂ for about 30 minutes to about 2 hours to provide a population of secondary transitional cells. In one aspect, the population of H₂O₂-evolved cells is contacted with about 15 mM to about 20 mM H₂O₂ for about 30 minutes to about 2 hours. In one aspect, the concentration of H₂O₂ used in step (f) is greater than the concentration of H₂O₂ used in step (c). In one aspect, the concentration of H₂O₂ used in step (f) is at least about 3 mM, about 3.5 mM, about 4 mM, about 4.5 mM, or about 5 mM and up to about 6 mM, about 7 mM, about 8 mM, about 9 mM or about 10 mM greater than the concentration of H₂O₂ used in step (c). In one aspect, the population of cells is contacted with about 18 mM, about 18.5 mM, or about 19 mM H₂O₂ for about 1 hour;
(g) resuspending the secondary transitional cells in fresh chemically defined culture media that does not include H₂O₂ and culturing until cells reach at least about 90% viability;
(h) incubating the secondary transitional cells with about 20 mM to about 40 mM H₂O₂ for about 30 minutes to about 2 hours to obtain a population of H₂O₂-evolved host cells. In one aspect, the secondary transitional cells are incubated with about 35 mM to about 40 mM H₂O₂ for about 1 hour; and
(i) resuspending the H₂O₂-evolved host cells in fresh chemically defined culture media that does not include H₂O₂ and culturing until cells reach at least about 90% viability.

In one aspect, providing a population of cells includes providing suspension adapted host cells. In one aspect, a suspension adapted CHO cells are provided. In one aspect, suspension adapted CHO-K1 cells are provided.

In one aspect, culturing the population of cells in (b) includes culturing the cells in suspension in chemically defined cell culture media. In one aspect, the chemically defined cell culture media is supplemented with glutamine. In one aspect, the chemically defined cell culture media is supplemented with L-glutamine. In one aspect, the chemically defined cell culture media is supplemented with about 2 mM to about 10 mM L-glutamine. In one aspect, the chemically defined cell culture media is supplemented with at least about 2 mM, about 3 mM, about 4 mM, or about 5 mM and up to about 6 mM, about 7 mM, about 8 mM, about 9 mM or about 10 mM L-glutamine. In one aspect, the chemically defined cell culture media is supplemented with about 2 mM, about 3 mM, about 4 mM, about 5 mM, about 6 mM, about 7 mM, about 8 mM, about 9 mM or about 10 mM L-glutamine. In one aspect, the population of cells in (b) is cultured to at least about 70%, about 80% or about 90% viability.

In one aspect, the population of cells in (c) is contacted with about 5 mM to about 10 mM, about 10 mM to about 15 mM, or about 15 to about 20 mM H₂O₂. In one aspect, the population of cells in (c) is contacted with about 10 mM, about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, or about 19.5 mM and up to about 20 mM H₂O₂. In one aspect, contacting in (c) comprises incubating the population of cells with H₂O₂ for at least about 30 min, about 45 min or about 60 min and up to about 90 min, or about 120 min.

In one aspect, culturing the population of cells in (d) includes culturing the cells in suspension in chemically defined cell culture media. In one aspect, the chemically defined cell culture media is supplemented with glutamine. In one aspect, the chemically defined cell culture media is supplemented with L-glutamine. In one aspect, the chemically defined cell culture media is supplemented with about 2 mM to about 10 mM L-glutamine. In one aspect, the chemically defined cell culture media is supplemented with at least about 2 mM, about 3 mM, about 4 mM, or about 5 mM and up to about 6 mM, about 7 mM, about 8 mM, about 9 mM or about 10 mM L-glutamine. In one aspect, the chemically defined cell culture media is supplemented with about 2 mM, about 3 mM, about 4 mM, about 5 mM, about 6 mM, about 7 mM, about 8 mM, about 9 mM or about 10 mM L-glutamine. In one aspect, the population of cells in (d) is cultured to at least about 70%, about 80% or about 90% viability.

In one aspect, steps (c)-(d) are repeated at least about 3, 4, or 5 times. In one aspect, the population of cells in (c) is contacted with the same amount of H₂O₂ for the same amount of time each time step (c) is repeated. In one aspect, the population of cells in (c) is contacted with a different amount of H₂O₂ one or more of the times step (c) is repeated. In one aspect, the population of cells in (c) is contacted with H₂O₂ for a different amount of time one or more of the times step (c) is repeated. In one aspect, the population of cells in (c) is contacted with a higher concentration of H₂O₂ the final time step (c) is performed as compared to one or more of the previous times step (c) is performed. In one aspect, the population of cells in (c) is contacted with about 10 mM to about 15 mM H₂O₂ the first time step (c) is performed. In one aspect, the population of cells in (c) is contacted with about 10 mM, about 10.5 mM, about 11 mM, about 11.5 mM, about 12 mM, about 12.5 mM, about 13 mM, about 13.5 mM, about 14 mM, about 14.5 mM or about 15 mM H₂O₂ the first time step (c) is performed. In one aspect, the population of cells in (c) is contacted with about 15 mM to about 20 mM H₂O₂ the final time step (c) is performed. In one aspect, the population of cells in (c) is contacted with about 15 mM, about 15.5 mM, about 16 mM, about 16.5 mM, about 17 mM, about 17.5 mM, about 18, about 18.5 mM, about 19, about 19.5 mM, or to about 20 mM H₂O₂ the final time step (c) is performed.

In one aspect, the population of evolved cells obtained after step (e) can survive when challenged with about 20 mM to about 40 mM H₂O₂ after at least about 20, about 30, about 40, about 50, about 60, about 70, about 80, about 90, or about 100 and up to about 120 population doublings (PDL). In one aspect, the population of evolved cells can survive when challenged with about 30 mM to about 40 mM H₂O₂ after at least about 20, about 30, about 40, about 50, about 60, about 70, about 80, about 90, or about 100 and up to about 120 population doublings (PDL). In one aspect, the population of evolved cells can survive when challenged with about 35 mM to about 40 mM H₂O₂ after at least about 20, about 30, about 40, about 50, about 60, about 70, about 80, about 90, or about 100 and up to about 120 population doublings (PDL). In one aspect, the cell culture media includes from about 1 mM to about 10 mM, or about 2 mM to about 8 mM, or about 4 mM to about 6 mM L-glutamine.

In one aspect, the host cell is stably transfected or otherwise engineered to express a heterologous protein of interest. In one aspect, a host cell with improved viability in the presence of hydrogen peroxide (H₂O₂) is provided. In one aspect, the host cell has a viability of at least about 10%, about 15%, about 20%, about 25%, about 30%, about 35%, about 40%, about 45%, or about 50% viability after about 1 to about 15 days after exposure to about 2 mM to about 50 mM, about 10 mM to about 40 mM, about 20 mM to about 40 mM H₂O₂, about 30 mM to about 40 mM H₂O₂, or about 35 mM to about 40 mM H₂O₂ for at least about 30 minutes, 45 minutes, or 1 hour and up to about 1.5 hours or about 2 hours. In one aspect, the host cell is contacted with at least about 10 mM, 15 mM, 20 mM, 25 mM, 30 mM, 35 mM and up to about 40 mM, 45 mM or 50 mM H₂O₂. In one aspect, the host cell is contacted with about 30 mM, 31 mM, 32 mM, 33 mM, 34 mM, 35 mM, 36 mM, 37 mM, 38 mM, 39 mM or 40 mM H₂O₂.

In one aspect, the host cell has a viability of at least about 10% after about 1 to about 15 days after exposure to about 20 mM to about 40 mM for about 30 minutes to about 2 hours. In one aspect, the host cell has a viability of at least about 15% after about 1 to about 15 days after exposure to about 20 mM to about 40 mM for about 30 minutes to about 2 hours. In one aspect, the host cell has a viability of at least about 20% after about 1 to about 15 days after exposure to about 20 mM to about 40 mM for about 30 minutes to about 2 hours. In one aspect, the host cell has a viability of at least about 25% after about 1 to about 15 days after exposure to about 20 mM to about 40 mM for about 30 minutes to about 2 hours. In one aspect, the host cell has a viability of at least about 30% after about 1 to about 15 days after exposure to about 20 mM to about 40 mM for about 30 minutes to about 2 hours. In one aspect, the host cell has a viability of at least about 35% after about 1 to about 15 days after exposure to about 20 mM to about 40 mM for about 30 minutes to about 2 hours. In one aspect, the host cell has a viability of at least about 40% after about 1 to about 15 days after exposure to about 20 mM to about 40 mM for about 30 minutes to about 2 hours. In one aspect, the host cell has a viability of at least about 45% after about 1 to about 15 days after exposure to about 20 mM to about 40 mM for about 30 minutes to about 2 hours. In one aspect, the host cell has a viability of at least about 50% after about 1 to about 15 days after exposure to about 20 mM to about 40 mM for about 30 minutes to about 2 hours.

In one aspect, the host cell has a viability of at least about 10% after about 1 to about 15 days after exposure to about 30 mM to about 40 mM for about 30 minutes to about 2 hours. In one aspect, the host cell has a viability of at least about 15% after about 1 to about 15 days after exposure to about 30 mM to about 40 mM for about 30 minutes to about 2 hours. In one aspect, the host cell has a viability of at least about 20% after about 1 to about 15 days after exposure to about 30 mM to about 40 mM for about 30 minutes to about 2 hours. In one aspect, the host cell has a viability of at least about 25% after about 1 to about 15 days after exposure to about 30 mM to about 40 mM for about 30 minutes to about 2 hours. In one aspect, the host cell has a viability of at least about 30% after about 1 to about 15 days after exposure to about 30 mM to about 40 mM for about 30 minutes to about 2 hours. In one aspect, the host cell has a viability of at least about 35% after about 1 to about 15 days after exposure to about 30 mM to about 40 mM for about 30 minutes to about 2 hours. In one aspect, the host cell has a viability of at least about 40% after about 1 to about 15 days after exposure to about 30 mM to about 40 mM for about 30 minutes to about 2 hours. In one aspect, the host cell has a viability of at least about 45% after about 1 to about 15 days after exposure to about 30 mM to about 40 mM for about 30 minutes to about 2 hours. In one aspect, the host cell has a viability of at least about 50% after about 1 to about 15 days after exposure to about 30 mM to about 40 mM for about 30 minutes to about 2 hours.

In one aspect, the host cell has a viability of at least about 10% after about 1 to about 15 days after exposure to about 35 mM to about 40 mM for about 30 minutes to about 2 hours. In one aspect, the host cell has a viability of at least about 15% after about 1 to about 15 days after exposure to about 35 mM to about 40 mM for about 30 minutes to about 2 hours. In one aspect, the host cell has a viability of at least about 20% after about 1 to about 15 days after exposure to about 35 mM to about 40 mM for about 30 minutes to about 2 hours. In one aspect, the host cell has a viability of at least about 25% after about 1 to about 15 days after exposure to about 35 mM to about 40 mM for about 30 minutes to about 2 hours. In one aspect, the host cell has a viability of at least about 30% after about 1 to about 15 days after exposure to about 35 mM to about 40 mM for about 30 minutes to about 2 hours. In one aspect, the host cell has a viability of at least about 35% after about 1 to about 15 days after exposure to about 35 mM to about 40 mM for about 30 minutes to about 2 hours. In one aspect, the host cell has a viability of at least about 40% after about 1 to about 15 days after exposure to about 35 mM to about 40 mM for about 30 minutes to about 2 hours. In one aspect, the host cell has a viability of at least about 45% after about 1 to about 15 days after exposure to about 35 mM to about 40 mM for about 30 minutes to about 2 hours. In one aspect, the host cell has a viability of at least about 50% after about 1 to about 15 days after exposure to about 35 mM to about 40 mM for about 30 minutes to about 2 hours.

In one aspect, an evolved host cell is provided that is produced by the method described herein. In one aspect, an H₂O₂-evolved host cell stably transfected with a heterologous gene encoding a protein of interest is provided. In one aspect, the host cell has a viability of at least about 50%, about 55%, about 60%, about 65%, about 70%, about 75%, about 80% or about 85% within about 7 days, about 8 days, about 9 days, about 10 days, about 11 days, about 12 days, about 13 days or about 14 days post-transfection. In one aspect, the host cell has a viability from about 50% to about 85% at least about 7 days, about 8 days, about 9 days, about 10 days, about 11 days, about 12 days, about 13 days or about 14 days post-transfection. In one aspect, the host cell has a viability from about 50% to about 85% within about 7 days to about 14 days post-transfection. In one aspect, the H₂O₂-evolved host cell has a viability from about 70% to about 85% at least about 9 days, about 10 days, about 11 days, about 12 days, about 13 or about 14 days post-transfection. In one aspect, the host cell has a viability from about 70% to about 85% within about 9 days to about 12 days post-transfection.

In one aspect, the H₂O₂-evolved host cell has viable cell density (VCD) of at least about 0.5×10⁶ cells/ml, about 0.6×10⁶ cells/ml, about 0.7×10⁶ cells/ml, about 0.8×10⁶ cells/ml, about 0.9×10⁶ cells/ml, about 1.0×10⁶ cells/ml, about 1.1×10⁶ cells/ml, about 1.2×10⁶ cells/ml, about 1.3×10⁶ cells/ml, about 1.4×10⁶ cells/ml, about 1.5×10⁶ cells/ml, about 1.6×10⁶ cells/ml, about 1.7×10⁶ cells/ml, about 1.8×10⁶ cells/ml, about 1.95×10⁶ cells/ml, or about 2.0×10⁶ cells/ml at least about 7 days, about 8 days, about 9 days, about 10 days, about 11 days, about 12 days, about 13 days or about 14 days post-transfection. In one aspect, the H₂O₂-evolved host cell has viable cell density (VCD) from about 0.5×10⁶ cells/ml to about 2.0×10⁶ cells/ml at least about 7 days, about 8 days, about 9 days, about 10 days, about 11 days, about 12 days, about 13 days or about 14 days post-transfection. In one aspect, the H₂O₂-evolved host cell has viable cell density (VCD) from about 1.0×10⁶ cells/ml to about 1.6×10⁶ cells/ml at least about 9 days, 10 days, 11 days, about 12 days, about 13 days or about 14 days post-transfection.

### G. Cell Culture

In one aspect, a method is provided for producing a protein of interest, which includes culturing a H₂O₂-evolved host cells as described herein, harvesting the H₂O₂-evolved host cells, and purifying the protein of interest from the cells or cell culture media.

In one aspect, a method of culturing a host cell is provided. In one aspect, a method of culturing an evolved host cell is provided. In one aspect, a method of culturing a H₂O₂-evolved host cell is provided. In one aspect, the host cell is a mammalian host cell. In one aspect, the host cell is a Chinese hamster ovary (CHO) cell. In one aspect, the host cell is a H₂O₂-evolved Chinese hamster ovary (CHO) cell. In one aspect, the host cell is a recombinant host cell. In one aspect, the host cell is engineered to recombinantly express a protein of interest. In one aspect, the host cell is a H₂O₂-evolved host cell that is engineered to recombinantly express a protein of interest. In one aspect, the host cell is a H₂O₂-evolved CHO cell that is engineered to recombinantly express a protein of interest.

In one aspect, the host cell is cultured under conditions that allow for expression of a protein of interest. In one aspect, the host cell is cultured under conditions that allow for expression of a heterologous protein. In one aspect, the host cell is cultured under conditions that allow for expression of a heterologous antibody or antigen binding antibody fragment. In one aspect, the host cell is cultured under conditions that allow for expression of a bispecific antibody.

In one aspect, the evolved host cell demonstrates improved performance when expressing a protein of interest in cell culture as compared to a non-evolved control. In one aspect, the evolved host cell demonstrates improved performance when expressing an antibody or antigen binding antibody fragment in cell culture as compared to a non-evolved control. In one aspect, the evolved host cell demonstrates improved performance when expressing a difficult to express protein in cell culture as compared to a non-evolved control. In one aspect, the evolved host cell demonstrates improved performance when expressing a bispecific antibody in cell culture as compared to a non-evolved control. In one aspect, the host cell is an H₂O₂-evolved host cell that demonstrates improved performance in cell culture as compared to a non-evolved control. In one aspect, the H₂O₂-evolved host cell demonstrates improved performance under conditions of oxidative stress as compared to a non-evolved control.

In one aspect, an evolved host cell is cultured to produce a protein of interest. Suitable culture conditions for mammalian cells are known in the art and can include, for example, batch, fed batch, continuous, semi-continuous, and perfusion mode. In one aspect, the host cell is cultured in suspension. The host cell can be cultured in any suitable bioreactor, including, for example, a fluidized bed bioreactor, hollow fiber bioreactor, roller bottle, shake flasks, or stirred tank bioreactors. The host cell can be cultured with or without microcarrier.

The evolved host cell can be cultured at any scale of culture, from individual flasks and shaker flasks or wave bags, to one-liter bioreactors, and up to large scale industrial bioreactors. In one aspect, the evolved host cell is cultured in a small-scale culture, for example, from 125 ml to 500 ml. In one aspect, the evolved host cell is cultured in a large-scale process, for example, in a volume from about 100 liters to about 20,000 liters or more. Large-scale or commercial-scale cell cultures, for example, for the production of a therapeutic protein, can be maintained for days, or even weeks. During this time, the culture can be supplemented with a concentrated feed medium that contains components, such as nutrients and amino acids, which are consumed during the course of the culture. In one aspect, the media is supplemented at intervals during cell culture according to a fed-batch process.

Methods for separating cells from cell culture medium are known and include, but are not limited to, methods such as filtration, cell encapsulation, cell adherence to microcarriers, cell sedimentation or centrifugation. In one aspect, the protein of interest is purified from the cells or cell culture media. Protein purification strategies are known. For example, soluble proteins such as antibodies, antibody-binding fragments and bispecific antibodies, can be purified using commercially available concentration filters, and subsequently affinity purified using known methods, such as affinity resins, ion exchange resins, chromatography columns, and combinations thereof.

### H. Protein of Interest

In one aspect, the H₂O₂-evolved host cell is engineered to produce a protein of interest. In one aspect, the H₂O₂-evolved host cell is transfected with an expression vector that encodes the protein of interest. As used herein “transfection” refers to any process in which a polynucleotide is introduced into a cell. The H₂O₂-evolved host cell can be transfected with any suitable expression vector, including, but not limited to, chromosomal, non-chromosomal, and synthetic nucleic acid vectors. Examples of expression vectors include, but are not limited to, derivatives of SV40, bacterial plasmids, phage DNA, baculovirus, yeast plasmids, vectors derived from combinations of plasmids and phage DNA, and viral nucleic acid (RNA or DNA) vectors. In one aspect, the vector includes one or more genes encoding one or more polypeptide chains of an antibody or bispecific antibody. In one aspect, the H₂O₂-evolved host cell is stably transfected.

In one aspect, stable transfection includes electroporation followed by methionine sulphoximine (MSX) selection.

In one aspect, the protein of interest is a therapeutic protein. In one aspect, the protein of interest is an intracellular protein. In one aspect, the protein of interest is secreted. In one aspect, the protein of interest is a transmembrane protein. In one aspect, the therapeutic protein is a peptide therapeutic, for example, having a size of about 1 kDa to about 10 kDa. In one aspect, the protein of interest is a large molecule therapeutic, for example, having a size of greater than about 10 kDa, for example, from about 10 kDa to about 250 kDa. In one aspect, the protein of interest includes a single polypeptide chain. In one aspect, the protein of interest includes more than one polypeptide chain. In one aspect, the protein of interest is “difficult to express” (DTE). Examples of therapeutic proteins include, but are not limited to, antibodies, enzymes, hormones, interferons, interleukins, growth factors, Fc fusion proteins, and receptors.

In one aspect, the protein of interest is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is a bispecific antibody.

A wide variety of bispecific antibody formats are known and include bispecific antibodies with or without an Fc region. Bispecific antibody formats can include a symmetric or an asymmetric architecture. In one aspect, the bivalent bispecific antibody includes one binding site for each antigen. In one aspect, the bispecific antibody is a tetravalent bispecific antibody and includes two binding sites for each antigen. In one aspect, the bispecific antibody has one binding site for a first antigen and 2 or 3 binding sites for a second other antigen. (See, Brinkmann and Kontermann (2017) “The making of bispecific antibodies.” MAbs. 9(2):182-212).

The number of chains needed to produce a bispecific antibody can vary. In one aspect, the bispecific antibody is a bispecific IgGs that includes four different polypeptide chains. In one aspect, the bispecific antibody includes a smaller number of chains, for example, 3, 2 or a single polypeptide chain.

In one aspect, H₂O₂-evolved host cells can be used for the production of antibodies. In one aspect, the H₂O₂-evolved host cells are used for the production of a monoclonal antibody or an antigen binding antibody fragment. In one aspect, the H₂O₂-evolved host cells are used for the production of a bispecific antibody. In one aspect, the antibody is a therapeutic antibody.

In one aspect, a protein of interest is provided. In one aspect, the protein of interest is expressed by an H₂O₂-evolved host cell described herein. In one aspect, a pharmaceutical composition is provided that includes a protein of interest expressed by an H₂O₂-evolved host cell described herein. In one aspect, the pharmaceutical composition includes a therapeutically effective amount of the protein of interest and a pharmaceutically acceptable carrier. In one aspect, the protein of interest is an antibody or an antigen binding antibody fragment. In one aspect, the protein of interest is a bispecific antibody.

### I. Incorporation by Reference

All references cited herein, including patents, patent applications, papers, text books and the like, and the references cited therein, to the extent that they are not already, are hereby incorporated herein by reference in their entirety.

### WORKING EXAMPLES

Production of bispecific antibodies by manufacturing cell lines is often hindered by low product yields (Spiess et al. (2015) “Alternative molecular formats and therapeutic applications for bispecific antibodies”. Mol Immunol. 67(2 Pt A):95-106). In some cases, the low product yields are associated with cellular stress. For example, cellular redox state can be changed due to generation of reactive oxygen species (ROS) attributable to a complex interplay between the endoplasmic reticulum (ER) and mitochondria (Turrens J F. (2003) “Mitochondrial formation of reactive oxygen species”. J Physiol. 552(Pt 2):335-44; Templeton et al. (2013) “Peak antibody production is associated with increased oxidative metabolism in an industrially relevant fed-batch CHO cell culture”. Biotechnol Bioeng. 110(7):2013-24; Tu and Weissman (2004) “Oxidative protein folding in eukaryotes: mechanisms and consequences”. J Cell Biol. 164(3):341-6). Fluctuations in bioreactor conditions can result in changes in dissolved oxygen concentrations (Handlogten et al. (2020) “Online Control of Cell Culture Redox Potential Prevents Antibody Interchain Disulfide Bond Reduction”. Biotechnol Bioeng. 117(5):1329-1336; Handlogten et al. (2018) “Intracellular response of CHO cells to oxidative stress and its influence on metabolism and antibody production”. Biochemical Engineering Journal. 133:12-20) and cell culture medium components (Halliwell B. (2014) “Cell culture, oxidative stress, and antioxidants: avoiding pitfalls”. Biomed J. 37(3):99-105; Kelts et al. (2015) “Altered cytotoxicity of ROS-inducing compounds by sodium pyruvate in cell culture medium depends on the location of ROS generation”. Springerplus. 4:269; Schnellbaecher et al. (2019) “Vitamins in cell culture media: Stability and stabilization strategies”. Biotechnol Bioeng. 116(6):1537-55). Accumulation of ROS can damage the cells, resulting in inferior cell performance and reduced antibody titer. Described herein is a novel hydrogen peroxide (H₂O₂) evolved host that was generated and evaluated for expression of two industrially relevant bispecific antibodies (BisAbs).

### Example 1. CHO Host Cell Evolution Using Hydrogen Peroxide

Chinese hamster ovary (CHO) host cells were evolved to improve resistance to oxidative stress through multiple rounds of successive H₂O₂ exposure followed by recovery until the host cells demonstrated survival in the presence of 37 mM H₂O₂ (FIG. 1A). Specifically, CHO-K1 cells adapted to grow in suspension culture (CHO Control) were revived in chemically-defined (CD) CHO medium (Life technologies, Paisley, UK) supplemented with 6 mM L-glutamine (Life technologies, Paisley, UK) and passaged three times at 0.2×10⁶ cells/ml in a 30 ml culture volume. When cells reached 99% viability, they were incubated for 1 hour with 14 mM H₂O₂ (Sigma-Aldrich, UK) before centrifugation at 130 g for 5 minutes and resuspended in 30 ml fresh CD CHO supplemented with 6 mM L-glutamine. CHO cells were left to recover until cells reached 70% viability. This process was repeated an additional three times resulting in a total of four exposures to 14 mM H2O2, in which cells gradually recovered over a period of 10-20 days, with the medium being replenished periodically, until cells reached 70% viability. The cells were then subjected to one round of 18.5 mM H₂O₂ treatment following the method above until cells reached 90% viability. Finally, the cells were exposed to 37 mM H₂O₂ for 1 hour before centrifugation at 130 g for 5 minutes and resuspension in 30 ml fresh CD CHO supplemented with 6 mM L-glutamine and incubated until >90% viable. During this incubation, the culture medium was replaced once on day 12, as detailed above, and the cells were diluted to 0.3×10⁶ cells/ml on day 21 with fresh medium to aid recovery.

To ensure that the evolutionary changes induced by H₂O₂ treatment were maintained, the cells were passaged to 90 population doublings (PDL) and subjected to re-challenge with 37 mM H₂O₂. The H₂O₂ evolved host cells demonstrated improved survival (˜50% viability) compared to CHO control cells that were similarly challenged with H₂O₂ (FIG. 1B) indicating long term heritable resistance to high concentrations of H₂O₂.

The H₂O₂ treated CHO cells were cryopreserved. All experiments were performed using this cryopreserved cell stock.

### Example 2. H₂O₂ Evolved CHO Cell Survival when Grown in the Presence of Redox Chemical Stressors

To confirm the H₂O₂ evolved host cell's ability to resist oxidative stress, the cells were evaluated for survival in response to several prooxidant chemical compounds selected to mimic bioreactor stressors: Menadione Sodium Bisulphite (MSB), Buthionine Sulfoximine (BSO), Mercaptosuccinic Acid (MS) and Cobalt chloride (CoCl). These chemicals affect a diverse subset of intracellular redox pathways such as those involved in GSH biosynthesis and turnover (BSO and MS) (Lee et al. (1992) “Effects of buthionine sulfoximine treatment on cellular glutathione levels and cytotoxicities of cisplatin, carboplatin and radiation in human stomach and ovarian cancer cell lines”. Korean J Intern Med. 7(2):111-7; Dunning et al. (2013) “Glutathione and antioxidant enzymes serve complementary roles in protecting activated hepatic stellate cells against hydrogen peroxide-induced cell death”. Biochim Biophys Acta. 1832(12):2027-34), intracellular ROS generation (MSB) (Beck et al. (2011) “Ascorbate/menadione-induced oxidative stress kills cancer cells that express normal or mutated forms of the oncogenic protein Bcr-Abl. An in vitro and in vivo mechanistic study”. Invest New Drugs. 29(5):891-900) and hypoxia-induced ROS production (CoCl) (Zou et al. (2001) “Cobalt chloride induces PC12 cells apoptosis through reactive oxygen species and accompanied by AP-1 activation”. J Neurosci Res. 64(6):646-53).

Chem stress assays were performed according to the manufacturer's instructions (ChemStress®, Valitacell Ltd, Ireland). In brief, CHO or H₂O₂ evolved host cells were seeded into Valitacell ChemStress plates (ChemStress®, Valitacell Ltd, Ireland) at 18,000 cells/well in 90 μl proprietary medium supplemented with 6 mM L-glutamine. A control well was incubated with medium alone. Plates were incubated for 72-hours in a static incubator at 36.5° C., 6% CO₂. Following this, 10 μl of neat PrestoBlue dye (Thermo Fisher Scientific, UK) was added to all wells before plates were mixed for 20 seconds and incubated for a further 30 minutes at 36.5° C., 6% CO₂. Plates were analyzed using a PHERAstar plate reader (BMG LABTECH, Ortenberg, Germany) with preconfigured protocols (excitation 560 nm, emission 590 nm). Data was analyzed, using the ValitaAPP software (Valitacell Ltd, Ireland).

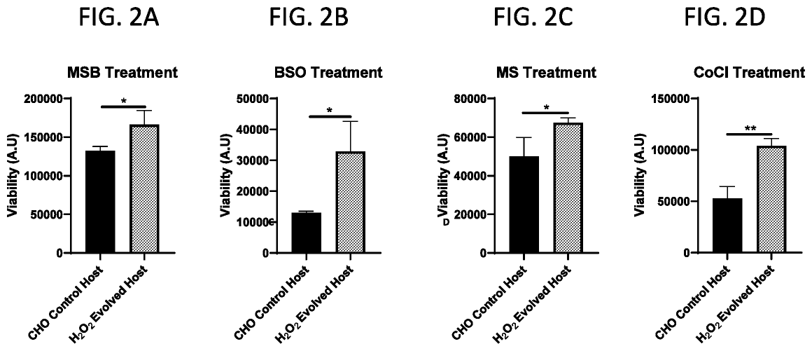

The H₂O₂ evolved host cells demonstrated significantly improved viabilities following 72 hours growth in the presence of MSB, BSO, MS and CoCl compared to CHO control cells (FIGS. 2A-2D) demonstrating that H₂O₂ evolved host cells had developed resistance to diverse redox stressors.

### Example 3. Antioxidant Capacity of H₂O₂ Evolved Cells

The ability of the H₂O₂ evolved host to survive in the presence of several redox stressors suggests that H₂O₂-induced evolution may have augmented antioxidant defense pathways within the cell. H₂O₂ evolved host and CHO control cells were therefore evaluated based on glutathione (GSH) content and transcriptional changes in a panel of antioxidant defense genes, which included genes involved in GSH synthesis (GSS and GCLM), H₂O₂ elimination (catalase) and cellular cysteine import (xCT).

Relative changes in intracellular total glutathione and oxidized glutathione (GSSG) were determined using the GSH/GSSG-Glo™ Assay kit (Promega, UK) according the manufacturer's instructions. Briefly, CHO Control or H₂O₂ evolved host cells in culture were harvested and resuspended in fresh proprietary medium supplemented with 6 mM L-glutamine (untransfected hosts) or 50 μM MSX (transfected pools). Cells were seeded at 10,000 cells/well in a white 96-well luminometer-compatible plate (medium only wells were used for background luminescence detection). A 25 μl volume of either total glutathione lysis reagent or oxidized glutathione lysis reagent was added to cell-containing wells and incubated at room temperature on a plate shaker for 5 minutes. 50 μl of freshly prepared luciferin generation reagent was added to all wells followed by a 30-minute incubation at room temperature. Finally, 100 μl of luciferin detection reagent was added to each well and incubated for 15 minutes. Luminescence was measured using the Perkin Elmer Envision Microplate Luminometer. Analysis was performed according to the manufacturer's instructions.

RNA was extracted from CHO control or H₂O₂ evolved host cells (both untransfected and transfected) using the Qiagen RNA isolation kit according to the manufacturer's instructions. cDNA was generated by reverse transcribing 3 μg of RNA using the SuperScript™ IV First-Strand Synthesis System (Thermo Fisher Scientific, UK) according to the manufacturer's instructions. qPCR probes are detailed in Table 1. qPCR was performed using the TaqMan Assay system (Thermo Fisher Scientific, UK) on the QuantStudio 12K Flex Real-time PCR System (Applied Biosciences, Massachusetts, USA). Relative gene expression was calculated using the delta Ct method.

TABLE 1
qPCR Probes

| Primer | Catalog No. | Reference No. |
|---|---|---|
| Catalase | 451372 | Cg04624486_m1 |
| GCLM | 451372 | Cg04497880_m1 |
| CPrx1 | 451372 | Cg04422105_g1 |
| GSS | 451372 | Cg04491342_m1 |
| xCT | 451372 | Cg04496729_m1 |
| MMADHC | 451372 | Cg04467875_m1 |

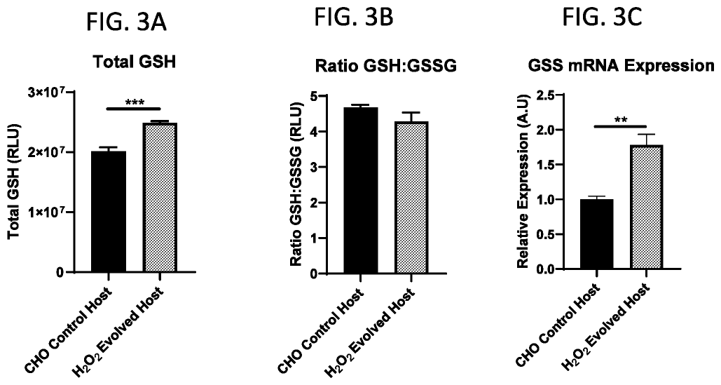

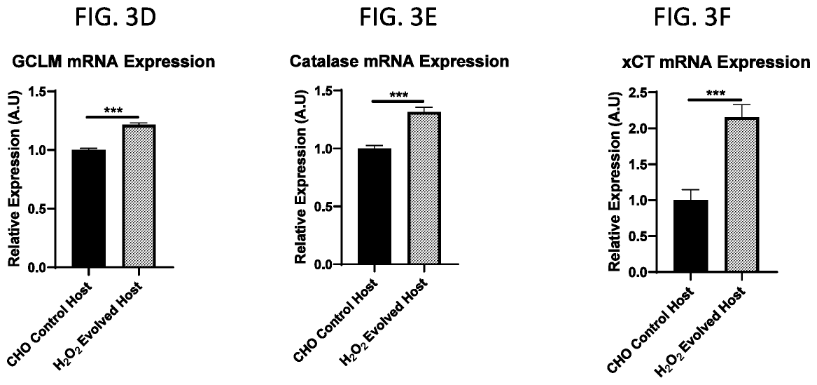

Early passage H₂O₂ evolved host cells were shown to have significantly elevated total glutathione content with respect to CHO control cells, although the ratio of total glutathione to oxidized GSH (GSH:GSSG) remained unchanged (FIGS. 3A and 3B). The H₂O₂ evolved host cells demonstrated significantly elevated expression of GSS and GCLM (FIGS. 3C and 3D), an observation consistent with the elevated total glutathione content of the H₂O₂ evolved host. Additionally, the H₂O₂ evolved host demonstrated significantly elevated expression of catalase (FIG. 3E) and xCT (FIG. 3F), indicating further improvements in antioxidant capacity.

Consistent with this improved ability to resist oxidative stress, the H₂O₂ evolved host was also found to significantly upregulate several central antioxidant defense genes that combat ROS through multiple mechanisms (FIG. 3C-3F). These genes include the H₂O₂ scavenger, catalase, along with serval enzymes associated with GSH production and activity (GCLM, GSS and xCT).

Upregulation in genes involved in GSH synthesis correlated with a significant increase in the level of total glutathione content in the H₂O₂ evolved host (FIG. 3A). Both the H₂O₂ evolved host and CHO control cells were able to maintain redox homeostasis through a preserved GSH:GSSG ratio when in the non-expressing state, possibly due to the absence of an oxidative stressor.

### Example 4. H₂O₂ Evolved Host Cells Expressing Bispecific Antibodies Maintain Resistance to Oxidative Stress

Expressing pools were compared for survival in the presence of the widely used prooxidant, menadione sodium bisulphite (MSB). Briefly, CHO Control or H₂O₂ evolved host cells expressing BisAbs A or BisAb B were seeded at 0.3×10⁶ cells/ml into 30 ml of proprietary medium supplemented with 50 μM MSX. A total of 6 μM Menadione Sodium Bisulfite (MSB) (Sigma-Aldrich, UK) or water was added to test and control cultures, respectively. Cells were assessed for viability and viable cell density (VCD) using a Vi-Cell XR Cell Viability Analyser (Beckman Coulter, USA) at 24, 48 and 72 hours post addition of MSB or water.

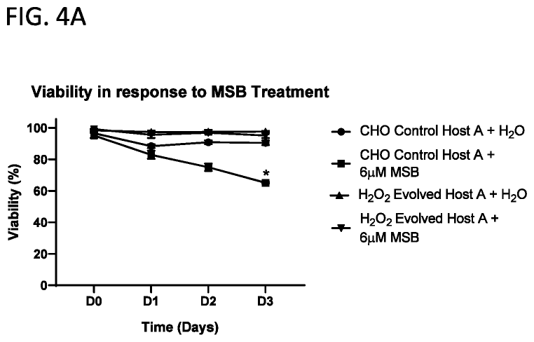

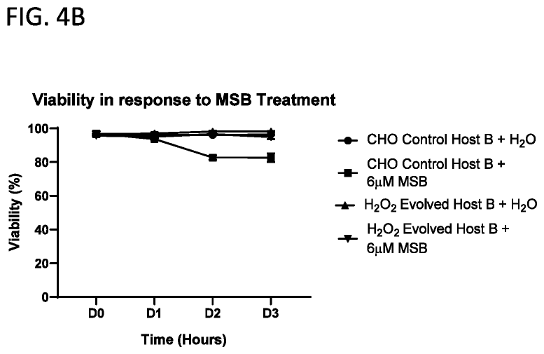

H₂O₂ evolved hosts A and B treated with MSB retained viabilities comparable with H₂O treated controls. By contrast CHO control hosts A and B treated with MSB displayed a reduction in viability at day 3 post-treatment to 65% and 82%, respectively (FIGS. 4A and 4B), indicating that the ability of the H₂O₂ evolved host to support improved expression of BisAbs may be linked to enhanced antioxidant capacity.

### Example 5. H₂O₂ Evolved Bispecific Antibody-Expressing Cells Maintain an Elevated Antioxidant Capacity

To investigate whether the H₂O₂ evolved host, when expressing bispecific antibodies, maintained resistance to oxidative stress, CHO control host cells and H₂O₂ evolved host cells, generated as described in Example 1, were stably transfected with stable expression plasmids for two industrially relevant bispecific antibodies—bispecific antibody A (BisAb A) and bispecific antibody B (BisAb B). The BisAb A and BisAb B expression plasmids were modified from transient expression plasmids (Daramola et al. (2014) “A high-yielding CHO transient system: coexpression of genes encoding EBNA-1 and GS enhances transient protein expression”. Biotechnol Prog. 30(1):132-41; Persic et al. (1997) “An integrated vector system for the eukaryotic expression of antibodies or their fragments after selection from phage display libraries”. Gene. 187(1):9-18) and encoded both the BisAb heavy chain (HC) and light chain (LC) in addition to a glutamine synthetase (GS) selectable marker (Lonza, Slough, UK). The plasmids were constructed using standard restriction enzyme digestion and ligation methods and transfected using an Amaxa nucleofector and reagents (Lonza, Cologne, Germany).

The transfected cells were selected and maintained in CD CHO or proprietary medium supplemented with 50 μM methionine sulfoxamine (MSX) (Sigma-Aldrich, Dorset, UK) in a humidified incubator at 36.5° C., 6% CO₂ with agitation at 140 rpm as required to yield stable pools (resulting transfected cell populations are denoted: H₂O₂ evolved host A or B and CHO control host A or B). Pools were counted regularly during transfection recovery using a Vi-Cell XR Cell Viability Analyser (Beckman Coulter, USA) and were expanded for use in the production of BisAb A and BisAb B in a 12-day fed-batch process using proprietary medium. The medium was supplemented with bolus additions of a proprietary nutrient feed added over the course of the culture period.

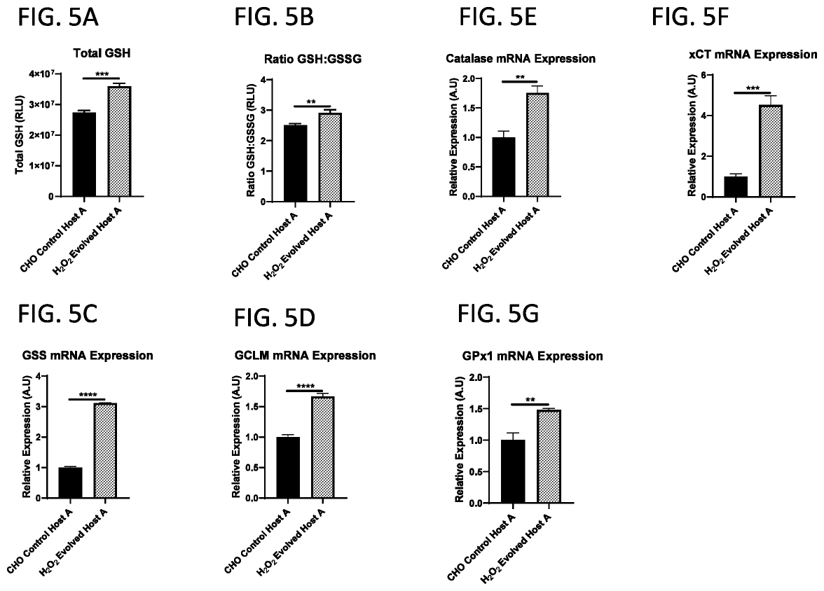

To investigate whether the elevated antioxidant phenotypes observed in the untransfected H₂O₂ evolved host cells were maintained in H₂O₂ evolved hosts A and B, both GSH content and antioxidant defense gene expression were assessed. When the H₂O₂ evolved host was placed under stressful conditions such as those induced by the expression of BisAbs, both H₂O₂ evolved hosts A and B displayed a significant upregulation in total GSH content as well as an improved ratio of total glutathione:GSSG as compared to CHO control cells (FIGS. 5A and 5B; 6A and 6B), although the increase was less apparent in H₂O₂ evolved host B.

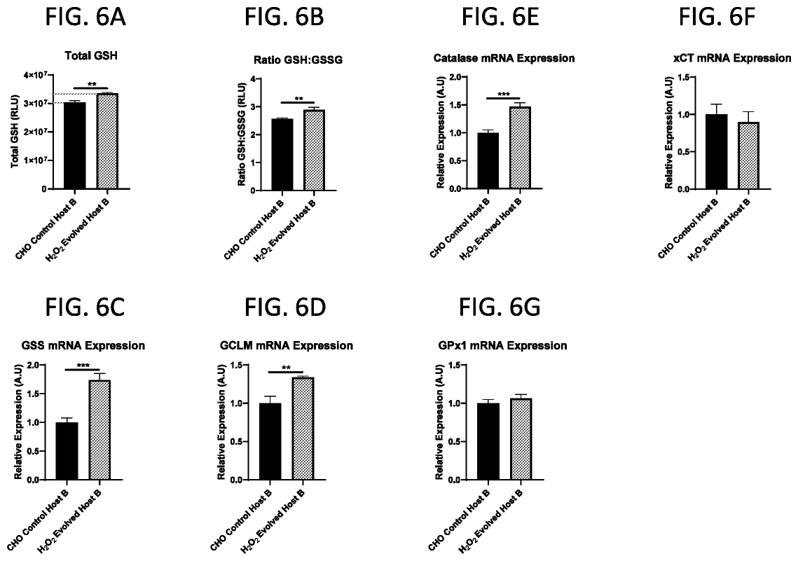

H₂O₂ evolved host pools were then assessed for expression of serval antioxidant defense genes. H₂O₂ evolved host A demonstrated significant elevations in GSS, GCLM, catalase and xCT as well as Glutathione peroxidase 1 (GPx1) compared to CHO control host A (FIG. 5C-5G). H₂O₂ evolved host B also showed significant elevations in GSS, GCLM and Catalase (FIG. 6C-6E). However, xCT and GPx1 remained unchanged with respect to CHO control Host B (FIGS. 6F and 6G).

Taken together, the data suggests that H₂O₂ directed evolution of the CHO host resulted in divergent expression of numerous antioxidant defense genes that renders the host capable of withstanding multiple sources of oxidative stress.

The H₂O₂ evolved host cells displayed significantly elevated catalase expression in both non-expressing and expressing states (FIGS. 3E, 5E and 6E). Although catalase activity was not measured, previous studies have demonstrated that catalase activity can be enhanced using directed host cell evolution (Spitz et al. (1988) “Stable H2O2-resistant variants of Chinese hamster fibroblasts demonstrate increases in catalase activity”. Radiat Res. 114(1):114-24) and may facilitate elimination of excessive H₂O₂ production to prevent oxidative stress.

Taken together these data indicate that the improvements in antioxidant capacity observed in the untransfected H₂O₂ evolved host (FIG. 3A-3F) are maintained upon expression of both bispecific antibodies, that the H₂O₂ evolved host is better equipped to combat excessive H₂O₂ production and that H₂O₂-induced evolution can improve cell performance through elevated antioxidant expression.

### Example 6. The H₂O₂ Evolved Host Demonstrated Improved Performance Compared to CHO Control Cells

To investigate whether the enhanced antioxidant capacity of the H₂O₂ evolved host translated to improved cell performance, the host cells were evaluated at two stages of a cell line generation process associated with elevated cellular stress: recovery from stable transfection by electroporation and during a fed-batch process.

a. H₂O₂ Evolved Host Cell Performance During Transfection Recovery

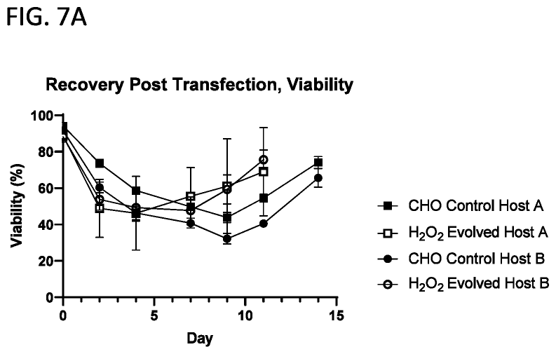

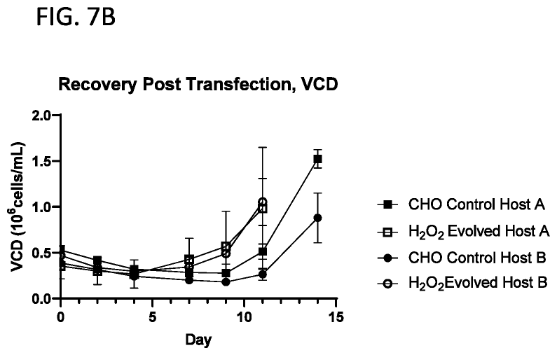

To evaluate the H₂O₂ host performance during transfection recovery, cell viability and viable cell density (VCD) were monitored following transfection. The H₂O₂ evolved host transfected with plasmid DNA encoding BisAb A reached viabilities of 82-84% and VCDs of 1.2-1.50×10⁶ cells/ml on day 11 post transfection and were transferred to shaking cultures. In contrast, CHO control host transfected with BisAb A reach a similar level of recovery (VCD 1.4-1.6×10⁶ cells/ml with viabilities of 71-78%, FIG. 7A) at day 14 post-transfection. The same trend was seen with BisAb B where H₂O₂ evolved host transfected pools reached 71-82% viabilities and VCDs of 0.88-1.35×10⁶ cells/ml on day 11 post transfection and the equivalent CHO control host transfected pools reaching 60-70% viability and 0.65-1.18×10⁶ cells/ml at day 14 before being transferred to shaking cultures (FIG. 7B). The data show that the H₂O₂ evolved host demonstrated faster recovery (11 days post transfection) compared to CHO control host cells (14 days post transfection) both in terms of viability and VCD (FIGS. 7A and 7B) following stable transfection with plasmid DNA encoding two distinct bispecific antibodies, indicating that the H₂O₂ evolved host has increased resistance to cellular stress compared to the CHO control cells.

b. H₂O₂ Evolved Host Cell Performance in a Fed Batch Process

To assess host cell performance during BisAb production, a 12-day fed batch process was performed with H₂O₂ evolved hosts A and B alongside CHO control hosts A and B.

Glucose and lactate were monitored throughout the fed-batch process using a YSI analyzer (2900D, YSI Inc). Cell culture medium was clarified by centrifugation and the BisAbs were quantified using protein-A high performance liquid chromatography (HPLC) affinity chromatography on an Agilent 1260 Infinity series HPLC system (Agilent Technologies, Santa Clara, CA) by comparing the peak size from each sample with a calibration curve.

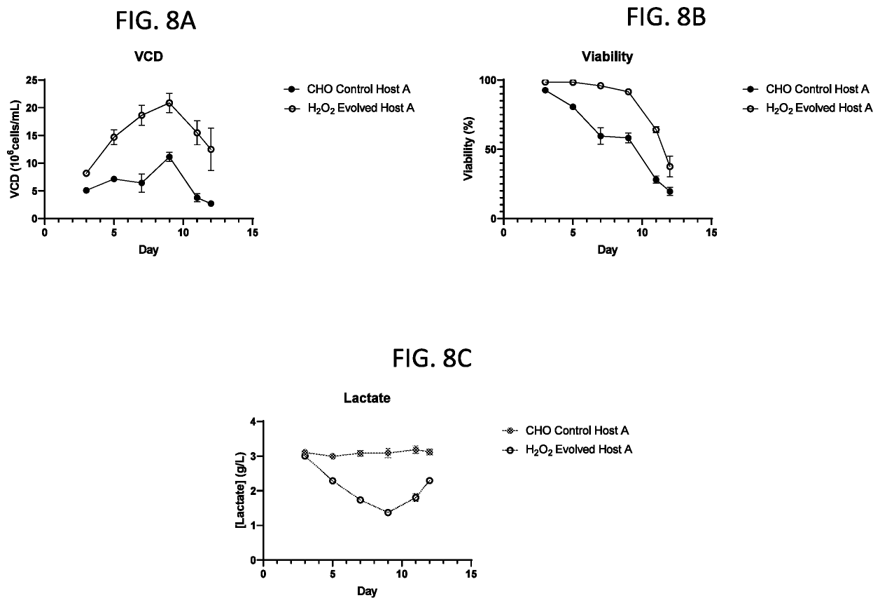

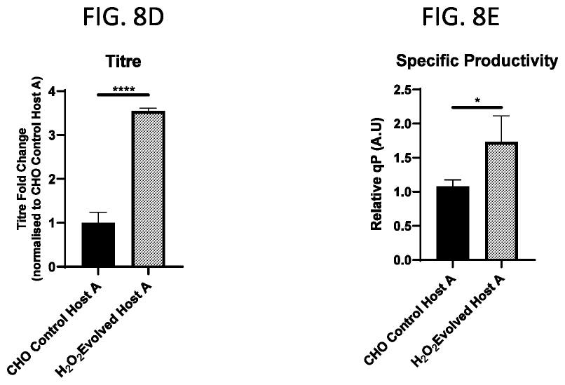

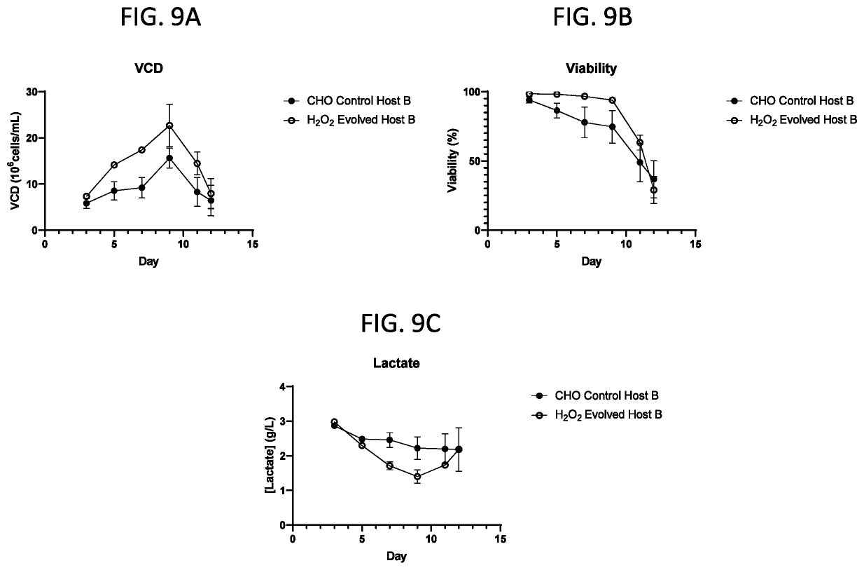

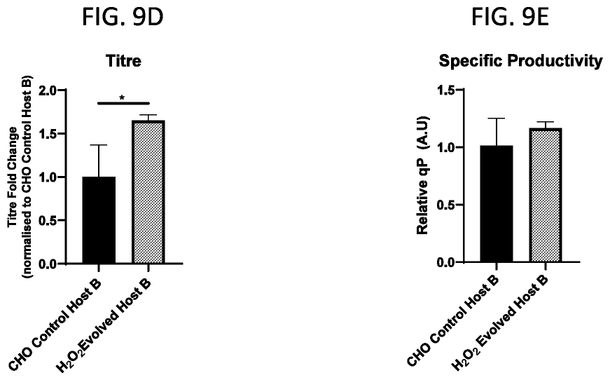

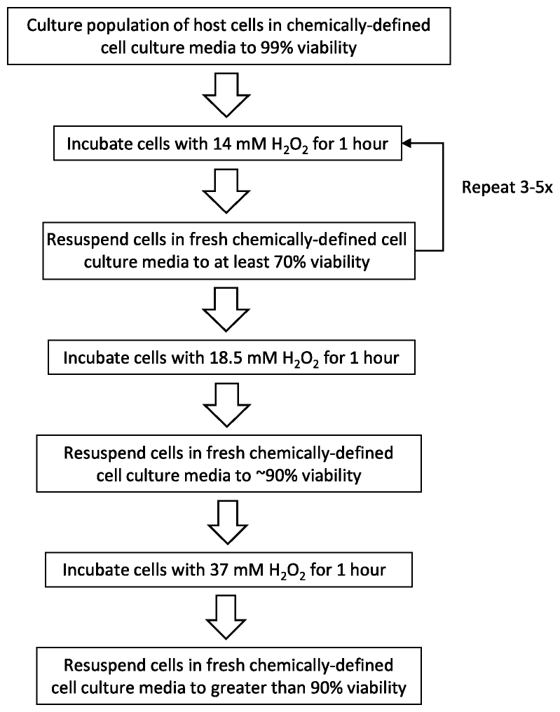

Growth rates of H₂O₂ evolved hosts A and B were dramatically improved compared to CHO control pools irrespective of the BisAb being expressed (FIGS. 8A and 9A). Peak VCD for H₂O₂ evolved host A was 20×10⁶ cells/ml compared to 7×10⁶ cells/ml for CHO control host A. Similar trends were also observed for H₂O₂ evolved host B. In addition, viabilities remained significantly higher for H₂O₂ evolved hosts A and B compared to CHO control hosts A and B, although viabilities at day 12 were comparably low between both hosts (FIGS. 8B and 9B).

All hosts displayed favorable lactate profiles, with H₂O₂ evolved hosts A and B having lower lactate levels throughout the majority of the fed-batch process (FIGS. 8C and 9C).

The titers of BisAb A were 3.5-fold higher from H₂O₂ evolved host A compared to the CHO control host (FIG. 8D). Improvements in titer were also seen for BisAb B where titers from the H₂O₂ evolved host were 1.75-fold higher than the CHO control host (FIG. 9D). Interestingly, H₂O₂ evolved host A demonstrated a significant increase in specific productivity (qP) of 1.7-fold compared to CHO control A (FIG. 8E). Indeed, the increased volumetric titer observed for BisAb A is likely derived from a combination of improved qP as well as cell growth and viability. By contrast the improved titer seen for BisAb B appears to result from improved growth and viability as qP was not significantly different between hosts (FIG. 9E).

### Example 7. H₂O₂ Evolved Host Expression of a Monoclonal Antibody (mAb)

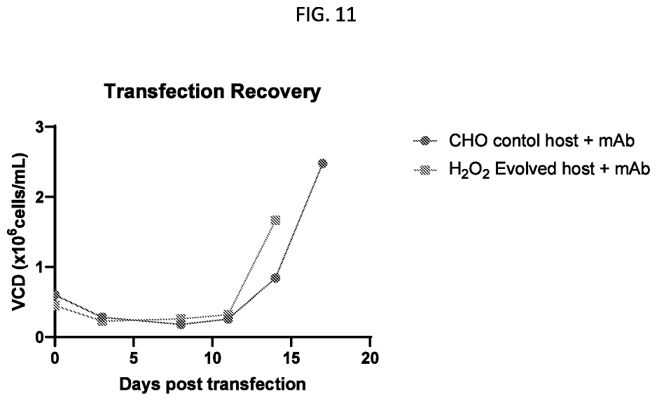

An H₂O₂-evolved CHO host cell, generated as described in Example 1, and a CHO parental control were transfected with linearized DNA encoding a murine IgG monoclonal antibody (mAb) to generate 1 pool of each. As shown in FIG. 11, the H₂O₂-evolved host cells recovered by day 14, whereas the parental control did not recover until day 17.

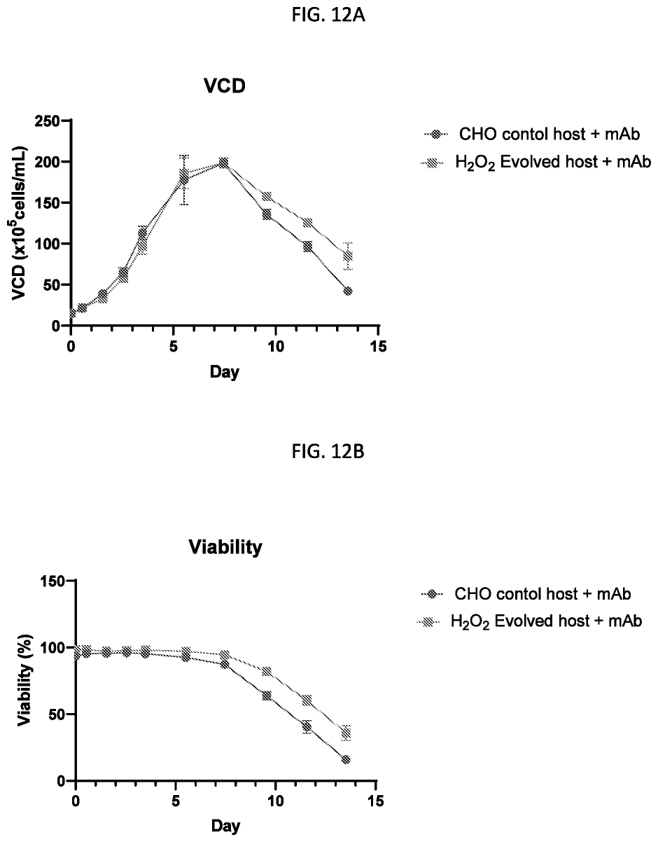

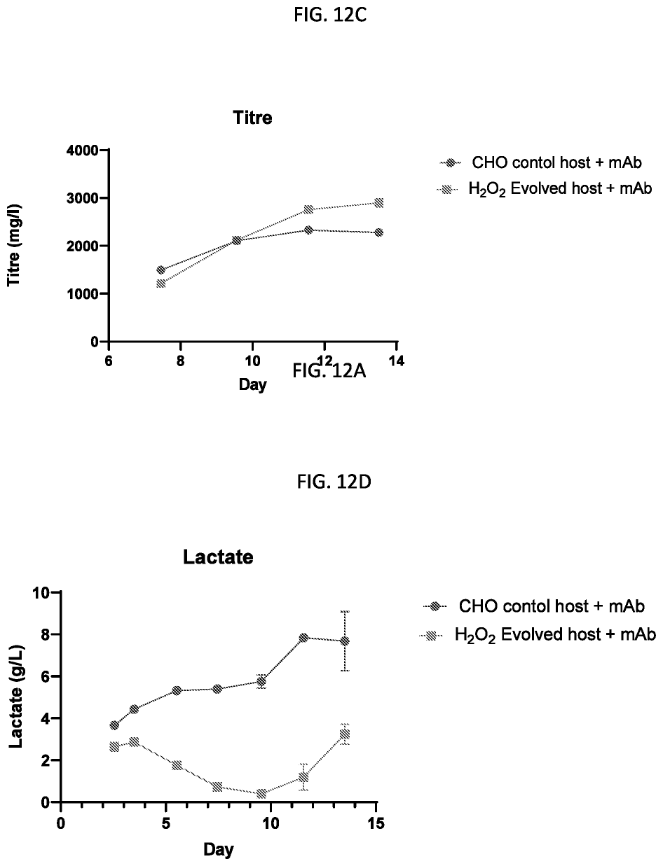

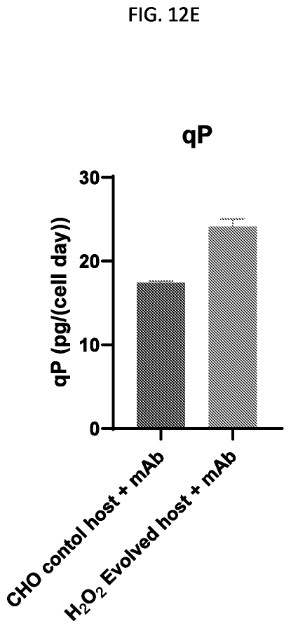

The performance of the H₂O₂-evolved host cells during production of the mAb was determined using an AMBR® high throughput, automated bioreactor. Each pool was inoculated into two vessels to generate 2 technical replicates. Performance parameters were monitored for 14 days following the methods described in Example 6(b). As shown in FIG. 12, the H2O2-evolved host showed similar growth to the CHO control (FIG. 12A), improved viability compared to the CHO control (FIG. 12B), an increase in titre compared to the CHO control (FIG. 12C), an improved lactate profile compared to the CHO control (FIG. 12D), and an increase in specific productivity compared to the CHO control (FIG. 12E).

1. A hydrogen peroxide (H₂O₂)-evolved mammalian host cell capable expressing a protein of interest, wherein the H₂O₂-evolved host cell has an increased resistance to cellular stress when compared to a parental control.

2. The H₂O₂-evolved host cell of claim 1, wherein the H₂O₂-evolved host cell has an antioxidant defense system in which a level of one or more components of the antioxidant defense system are increased when compared to a parental control.

3. The H₂O₂-evolved host cell of claim 2, wherein one or more components of the antioxidant defense system are selected from glutathione (GSH), oxidized glutathione (GSSG), glutathione synthetase (GSS); glutamate cysteine ligase modifier subunit (GCLM); catalase; cysteine/glutamate antiporter light chain (xCT); glutathione peroxidase-1 (GPx-1); and combinations thereof.

4. The H₂O₂-evolved host cell of claim 3, wherein the H₂O₂-evolved host cell has an increased level of GSH when compared to the parental control.

5. The H₂O₂-evolved host cell of claims 3 or 4, wherein the H₂O₂-evolved host cell has from about a 1% to about 25%, about 2% to about 20%, or about 3% to about 10%, or at least about 1%, about 2%, about 3%, about 4%, about 5%, about 6%, about 7%, about 8%, about 9%, about 10%, about 15%, or about 20% higher level of GSH than the parental control.

6. The H₂O₂-evolved host cell of any of claims 3 to 5, wherein the H₂O₂-evolved host cell has an increased ratio of total glutathione to oxidized glutathione (GSSG) (GSH:GSSG) when compared to a parental control.

7. The H₂O₂-evolved host cell of any of claims 3 to 6, wherein the ratio of total glutathione to GSSG (GSH:GSSG) is increased by about 1% to about 15%, or about 2% to about 10%, or at least about 1%, about 2%, about 3%, about 4%, about 5%, about 6%, about 7%, about 8%, about 9%, about 10%, or about 15% as compared to the parental control.

8. The H₂O₂-evolved host cell of claim 6 or 7, wherein the ratio of total glutathione to GSSG is from about 2.5:1 to about 3:1, or at least about 2.5:1, about 2.6:1, about 2.7:1, about 2.8:1, about 2.9:1 or about 3:1.

9. The H₂O₂-evolved host cell of any of the preceding claims, wherein one or more antioxidant defense genes are upregulated as compared to the parental control.

10. The H₂O₂-evolved host cell according to claim 9, wherein one or more antioxidant defense genes are selected from: glutathione synthetase (GSS); glutamate cysteine ligase modifier subunit (GCLM); catalase; cysteine/glutamate antiporter light chain (xCT); glutathione peroxidase-1 (GPx-1); and combinations thereof.

11. The H₂O₂-evolved host cell according to claim 10, wherein GSS expression is increased about 10% to about 300%, or about 25% to about 200%, or at least about 10%, about 20%, about 30%, about 40%, about 50%, about 60%, about 70%, about 80%, about 90%, about 100% and up to about 150%, about 200%, about 250% or about 300% as compared to the parental control.

12. The H₂O₂-evolved host cell according to claim 10 or 11, wherein GCLM expression is increased about 10% to about 100%, or about 25% to about 75%, or at least about 10%, about 15%, about 20%, about 25%, about 30%, about 35%, about 40%, about 45%, about 50%, about 55%, about 60%, about 65%, about 70%, or about 75% as compared to the parental control.

13. The H₂O₂-evolved host cell according to any of claims 10 to 12, wherein catalase expression is increased about 10% to about 100%, or about 25% to about 75%, or at least about 10%, about 15%, about 20%, about 25%, about 30%, about 35%, about 40%, about 45%, about 50%, about 55%, about 60%, about 65%, about 70%, or about 75% as compared to the parental control.

14. The H₂O₂-evolved host cell according to any of claims 10 to 13, wherein xCT expression is increased about 10% to about 50%, or at least about 10%, about 15%, about 20%, about 25%, about 30%, about 35%, about 40%, about 45%, or about 50% as compared to the parental control.

15. The H₂O₂-evolved host cell according to any of claims 10 to 14, wherein GPx-1 expression is increased about 10% to about 100%, or about 10% to about 50%, or at least about 10%, about 15%, about 20%, about 25%, about 30%, about 35%, about 40%, about 45%, or about 50% as compared to the parental control.

16. The H₂O₂-evolved host cell of any of the preceding claims, comprising a Chinese hamster ovary (CHO) cell.

17. The H₂O₂-evolved host cell of any of the preceding claims, comprising a heterologous gene encoding a protein of interest.

18. The H₂O₂-evolved host cell of any of the preceding claims, wherein the heterologous gene is stably integrated into host cell DNA.

19. The H₂O₂-evolved host cell of claim 17 or 18, wherein the heterologous gene encodes an antibody or antigen-binding antibody fragment.

20. The H₂O₂-evolved host cell of any of claims 17 to 19, wherein the heterologous gene encodes a bispecific antibody.

21. A hydrogen peroxide (H₂O₂)-evolved Chinese hamster ovary (CHO) cell expressing a therapeutic protein of interest, wherein the H₂O₂-evolved host cell has an antioxidant defense system in which a level of one or more components of the antioxidant defense system selected from glutathione (GSH), oxidized glutathione (GSSG), glutathione synthetase (GSS); glutamate cysteine ligase modifier subunit (GCLM); catalase; cysteine/glutamate antiporter light chain (xCT); glutathione peroxidase-1 (GPx-1); are increased when compared to a parental control.

22. A population of cells comprising the H₂O₂-evolved host cell of any of claims 1 to 21.

23. The population of cells of claim 22, wherein the H₂O₂-evolved cells have improved performance as compared to a population of parental control cells, wherein improved performance comprises one or more of:
(a) increased viable cell density;
(b) increased viability;
(c) decreased lactate levels;
(d) increased titer;
(e) increased specific productivity (qP);
or a combination thereof.

24. The population of cells of claim 23, wherein the H₂O₂-evolved cells have improved performance as compared to a population of parental control cells when cultured in a fed-batch culture process.

25. The population of cells of claim 23 or 24, wherein the H₂O₂-evolved cells have improved performance as compared to a population of parental control cells when expressing a protein of interest.

26. The population of cells of claim 25, wherein the protein of interest comprises a bispecific antibody.

27. The population of cells of any of claims 23 to 26, wherein the population of H₂O₂-evolved cells has an increased viable cell density as compared to the population of parental control cells.

28. The population of cells of any of claims 23 to 27, wherein the population of H₂O₂-evolved cells has a peak viable cell density from about 15×10⁶ cells/mL to about 25×10⁶ cells/mL, or about 15×10⁶ cells/mL, about 16×10⁶ cells/mL, about 17×10⁶ cells/mL, about 18×10⁶ cells/mL, about 19×10⁶ cells/mL or about 20×10⁶ cells/mL.

29. The population of cells of any of claims 23 to 28, wherein the population of H₂O₂-evolved cells has increased viability as compared to the population of parental control cells.

30. The population of cells of claim 29, wherein the population of H₂O₂-evolved cells has at least about 10%, about 20%, about 30%, about 40%, or about 50% increased viability compared to the population of parental control cells when challenged with hydrogen peroxide.

31. The population of cells of claim 30, wherein the population of H₂O₂-evolved cells are challenged with at least about 5 mM, about 10 mM, about 15 mM, about 20 mM, about 25 mM, about 30 mM, about 35 mM, or about 40 mM, hydrogen peroxide.

32. The population of cells of claim 30, wherein the population of H₂O₂-evolved cells are challenged with about 30 mM, about 31 mM, about 32 mM, about 33 mM, about 34 mM, about 35 mM, about 36 mM, about 37 mM, about 38 mM, about 39 mM, or about 40 mM hydrogen peroxide.

33. The population of cells of any of claims 23 to 32, wherein the population of H₂O₂-evolved cells have reduced lactate levels as compared to the population of parental control cells.

34. The population of cells of any of claims 23 to 33, wherein the population of cells express a protein of interest at a titer that is increased at least about 1.5 fold to 3.5 fold, or at least about 1.5 fold, about 1.75 fold, about 2.0 fold, about 2.25 fold, about 2.5 fold, about 2.75 fold, about 3.0 fold, about 3.25 fold, or about 3.5 fold as compared to the population of parental control cells.

35. The population of cells of claim 34, wherein the protein of interest is a bispecific antibody.

36. The population of cells of claim 35, wherein the titer of the bispecific antibody is from about 0.5 g/L and about 1.5 g/L, or about 0.5 g/L and about 1.1 g/L, or from about 0.50 g/L, about 0.55 g/L, about 0.60 g/L, about 0.65 g/L, about 0.70 g/L, about 0.75 g/L, about 0.80 g/L, about 0.85 g/L, about 0.90 g/L, about 0.95 g/L, and up to about 1.0 g/L, about 1.1 g/L, about 1.2 g/L, about 1.3 g/L, about 1.4 g/L, or about 1.5 g/L.

37. The population of cells of claim 35 or 36, wherein the titer of the bispecific antibody is at least about 0.5 g/L, about 0.6 g/L, about 0.7 g/L, about 0.8 g/L, about 0.9 g/L, or about 1.0 g/L.

38. The population of cells of any of claims 23 to 37, wherein the cells have a specific productivity (qP) that is increased at least about 1.1 fold, about 1.2 fold, about 1.3 fold, about 1.4 fold, about 1.5 fold, about 1.6 fold, about 1.7 fold, about 1.8 fold, about 1.9 fold, or about 2.0 fold as compared to the population of parental control cells.

39. A method of producing a protein of interest, comprising culturing a H₂O₂-evolved host cell according to any of claims 1 to 21 under conditions that allow for expression of the protein of interest.

40. The method of claim 39, wherein the protein of interest comprises a heterologous protein.

41. The method of claim 39 or 40, wherein the protein of interest comprises an antibody or an antigen-binding antibody fragment.

42. The method of claim 41, wherein the antibody comprises a bispecific antibody.

43. The method of any of claims 39 to 42, wherein the protein of interest is constitutively expressed.

44. The method of any of claims 39 to 43, further comprising isolating the protein of interest.

45. A composition comprising the isolated protein of interest of claim 44, and a pharmaceutically acceptable carrier.

46. Use of a H₂O₂-evolved host cell according to any of claims 1 to 21 for producing a protein of interest.

47. The use of claim 46, wherein the protein of interest comprises an antibody or an antigen-binding antibody fragment.

48. The use of claim 47, wherein the antibody comprises a bispecific antibody.

49. An isolated host cell with improved viability in the presence of hydrogen peroxide (H₂O₂), wherein the host cell has a viability of at least about 10%, about 15%, about 20%, about 25%, about 30%, about 35%, about 40%, about 45%, or about 50% viability after about 1 to about 15 days after about 30 minutes to about 2 hours exposure to about 2 mM to about 50 mM, about 10 mM to about 40 mM, about 20 mM to about 40 mM H₂O₂, about 30 mM to about 40 mM H₂O₂, or about 35 mM to about 40 mM H₂O₂.

50. The isolated host cell of claim 49, wherein the host cell is contacted with at least about 10 mM, about 15 mM, about 20 mM, about 25 mM, about 30 mM, about 35 mM and up to about 40 mM, about 45 mM or about 50 mM H₂O₂.

51. The isolated host cell of claim 49, wherein the host cell is contacted with about 30 mM, about 31 mM, about 32 mM, about 33 mM, about 34 mM, about 35 mM, about 36 mM, about 37 mM, about 38 mM, about 39 mM or about 40 mM H₂O₂.

52. A method of producing an evolved population of mammalian host cells with increased resistance to cellular stress, the method comprising multiple rounds of culturing the population of cells in the presence of about 5 mM to about 20 mM, or about 10 mM to about 15 mM hydrogen peroxide (H₂O₂) and allowing the cells to recover until cells can survive in the presence of from about 20 mM to about 40 mM H₂O₂.

53. The method of claim 52, comprising:
(a) providing a population of cells;
(b) culturing the population of cells in a cell culture media;
(c) contacting the population of cells with about 5 mM to about 20 mM H₂O₂ for about 30 minutes to about 120 minutes to provide a population of transitional cells;
(d) resuspending the transitional cells in fresh cell culture media that does not include H₂O₂ and culturing until cells reach at least about 70% viability;
(e) repeating steps (c)-(d) to obtain a population of H₂O₂-evolved cells that can survive when contacted with about 20 mM to about 40 mM H₂O₂ and incubated for about 30 minutes to about 1 hour.

54. The method of claim 52 or 53, wherein the population of cells comprise Chinese hamster ovary (CHO) cells.

55. The method of any of claims 52 to 54, wherein the cells comprise a heterologous gene encoding a protein of interest.

56. The method of claim 55, wherein the heterologous gene is stably integrated into host cell DNA.

57. The method of claim 55 or 56, wherein the heterologous gene encodes an antibody or an antigen-binding antibody fragment.

58. The method of any of claims 55 to 57, wherein the heterologous gene encodes a bispecific antibody.

59. The method of any of claims 52 to 58, wherein the cells are cultured in suspension.

60. The method of claim 53, wherein the population of cells in (b) is cultured to at least about 70%, about 80% or about 90% viability.

61. The method of claim 53, wherein the population of cells in (c) is contacted with about 5 mM to about 10 mM, about 10 to about 15 mM, or about 15 to about 20 mM H₂O₂.

62. The method of claim 53, wherein the population of cells in (c) is contacted with about 10 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, or about 20 mM H₂O₂.

63. The method of claim 53, wherein contacting in (c) comprises incubating the population of cells with H₂O₂ for at least about 30 min, about 45 min or about 60 min and up to about 90 min, or about 120 min.

64. The method of claim 53, wherein steps (c)-(d) are repeated at least about 3, 4, or 5 times.

65. The method of claim 53, wherein the population of evolved cells can survive when contacted with about 20 mM to about 40 mM H₂O₂ after at least about 20, about 30, about 40, about 50, about 60, about 70, about 80, about 90, or about 100 and up to about 120 population doublings (PDL).

66. The method of any of claims 53 to 65, wherein the cell culture media comprises from about 1 mM to about 10 mM, or about 2 mM to about 8 mM, or about 4 mM to about 6 mM L-glutamine.

67. An evolved host cell produced by the method of any of claims 52 to 66.

68. An H₂O₂-evolved host cell stably transfected with a heterologous gene encoding a protein of interest, wherein the host cell has a viability from about 70% to about 85% within about 9 to about 12 days post-transfection.

69. The H₂O₂-evolved host cell of claim 68, wherein the host cell has a viability from about 70% to about 85% at least about 9 days, about 10 days, about 11 days or about 12 days post-transfection.

70. The H₂O₂-evolved host cell of claim 68 or 69, wherein the host cell has viable cell density (VCD) from about 1.0×10⁶ cells/ml to about 1.6×10⁶ cells/ml at least about 9 days, 10 days, 11 days or 12 days post-transfection.

71. The H₂O₂-evolved host cell of any of claims 68 to 70, comprising a Chinese hamster ovary (CHO) cell.

72. The H₂O₂-evolved host cell of any of claims 68 to 71, wherein stable transfection comprises electroporation followed by methionine sulphoximine (MSX) selection.

73. The H₂O₂-evolved host cell of any of claims 68 to 72, wherein the heterologous gene encodes an antibody or an antigen-binding fragment.

74. The H₂O₂-evolved host cell of any of claims 68 to 73, wherein the heterologous gene encodes a bispecific antibody.
